(19) United States
(12) Patent Application Publication
(10) Pub. No.: US 2023/0109865 A1
(43) Pub. Date: Apr. 13, 2023

(54) Chemical Process for the Preparation of Herbicidal Pyridazine Compounds

(71) Applicant: SYNGENTA CROP PROTECTION AG, Basel (CH)

(72) Inventors: Raphael DUMEUNIER, Stein (CH); Tomas SMEJKAL, Stein (CH); Edouard GODINEAU, Stein (CH)

(73) Assignee: SYNGENTA CROP PROTECTION AG, Basel (CH)

(21) Appl. No.: 17/759,236

(22) PCT Filed: Jan. 20, 2021
(86) PCT No.: PCT/EP2021/051128

§ 371 Date: Jul. 22, 2022

(30) Foreign Application Priority Data
Jan. 21, 2020 (GB) 2000878.5

(51) Int. Cl. C07D 403/04; C07D 401/04; C07D 405/14; C07D 493/18

(52) U.S. Cl. CPC C07D 403/04; C07D 401/04; C07D 405/14; C07D 493/18

(57) ABSTRACT

The present invention provides, inter alia, a process for producing a compound of formula (I) wherein the substituents are as defined in claim 1, comprising reacting a compound of formula (II) in a suitable reaction medium comprising a desulfurization agent formula (II). The present invention further provides intermediate compounds utilised in said process, and methods for producing said intermediate compounds.

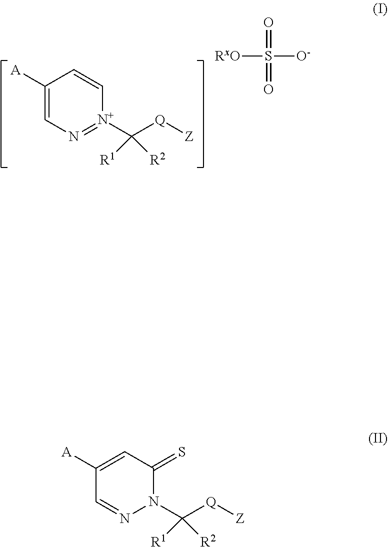

The present invention relates to a novel process for the synthesis of herbicidal pyridazine compounds. Such compounds are known, for example, from WO 2019/034757 and processes for making such compounds or intermediates thereof are also known. Such compounds are typically produced via an alkylation of a pyridazine intermediate.

The alkylation of pyridazine intermediates is known (see for example WO 2019/034757), however, such a process has a number of drawbacks. Firstly, this approach often leads to a non-selective alkylation on either pyridazine nitrogen atom and secondly, an additional complex purification step is required to obtain the desired product. Thus, such an approach is not ideal for large scale production and therefore a new, more efficient synthesis method involving a selective alkylation is desired to avoid the generation of undesirable by-products.

Surprisingly, we have now found that such a non-selective alkylation can be avoided by alkylation on an oxo-pyridazine which in turn can be converted to a thio-pyridazine and then further converted to the desired herbicidal pyridazine compounds. Such a process is more convergent, which may be more cost effective and may produce less waste products.

Thus, according to the present invention there is provided a process for the preparation of a compound of formula (I)

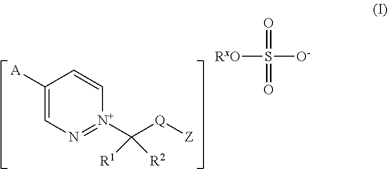

wherein
A is a 6-membered heteroaryl selected from the group consisting of formula A-I to A-VII below

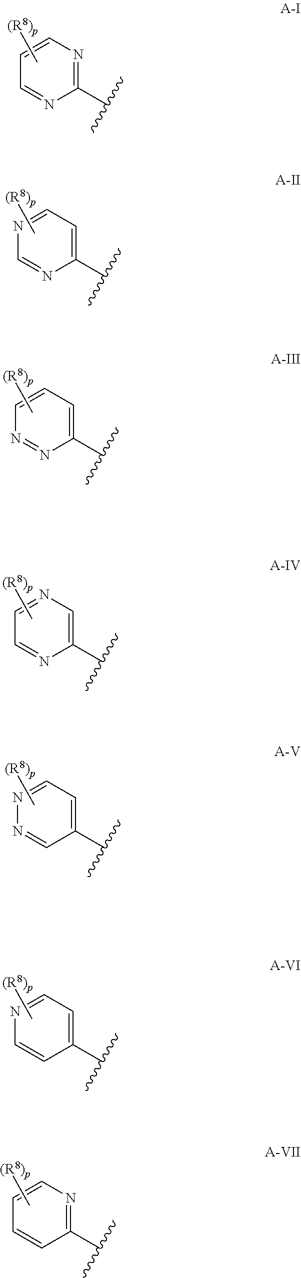

wherein the jagged line defines the point of attachment to the remaining part of a compound of formula (I), p is 0, 1 or 2; and
R^{x} is hydrogen or C₁-C₆alkyl;
R¹ is hydrogen or methyl;
R² is hydrogen or methyl;
Q is (CR^{1a}R^{2b})ₘ;
m is 0, 1 or 2;
each R^{1a} and R^{2b} are independently selected from the group consisting of hydrogen, methyl, —OH and —NH₂;
Z is selected from the group consisting of —CN, —C(S)OR¹⁰, —C(S)NR⁶R⁷, —C(S)SR¹⁰, —CH₂OR³, —CH(OR⁴)(OR^{4a}), —C(OR⁴)(OR^{4a})(OR^{4b}), —C(O)O R¹⁰, —C(O)NHCN, —C(O)NR⁶R⁷, —C(O)NHS(O)₂R¹² and —S(O)₂OR¹⁰; or
Z is selected from the group consisting of a group of formula Zₐ, Z_{b}, Z_{c}, Z_{d}, Zₑ and Z_{f} below

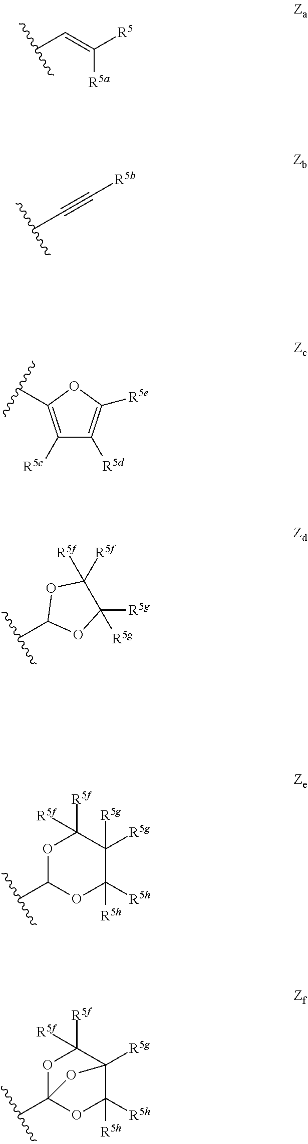

wherein the jagged line defines the point of attachment to the remaining part of a compound of formula (I); and
R³ is hydrogen or —C(O)OR^{10a};
each R⁴, R^{4a} and R^{4b} are independently selected from C₁-C₆alkyl;
each R⁵, R^{5a}, R^{5b}, R^{5c}, R^{5d}, R^{5e}, R^{5f}, R^{5g} and R^{5h} are independently selected from hydrogen and C₁-C₆alkyl;
each R⁶ and R⁷ are independently selected from hydrogen and C₁-C₆alkyl;
each R⁸ is independently selected from the group consisting of halo, —NH₂, methyl and methoxy;
R¹⁰ is selected from the group consisting of hydrogen, C₁-C₆alkyl, phenyl and benzyl;
R^{10a} is selected from the group consisting of hydrogen, C₁-C₆alkyl, phenyl and benzyl; and
R¹² is selected from the group consisting of methyl, —NH₂, —N(CH₃)₂ and —NHCH₃;
said process comprising:
reacting a compound of formula (II):

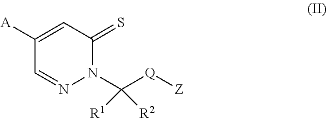

wherein A, R¹, R², Q and Z are as defined above, in a suitable reaction medium comprising a desulfurization agent, to give a compound of formula (I).

According to a second aspect of the invention, there is provided a compound of formula (I)

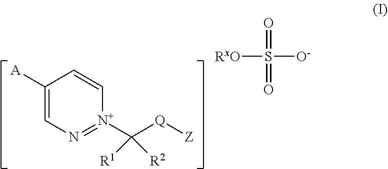

wherein A, R^{x}, R¹, R², Q and Z are as defined herein.

According to a third aspect of the invention, there is further provided an intermediate compound of formula (II):

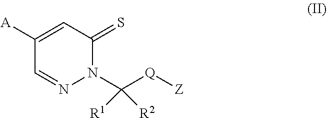

wherein A, R¹, R², Q and Z are as defined herein.

According to a fourth aspect of the invention, there is further provided an intermediate compound of formula (IV):

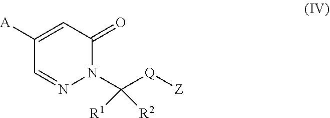

wherein A is a 6-membered heteroaryl selected from the group consisting of formula A-I to A-V and p, R¹, R², R⁸, Q and Z are as defined herein.

According to a fifth aspect of the invention, there is provided the use of a compound of formula (III-I) for preparing a compound of formula (I)

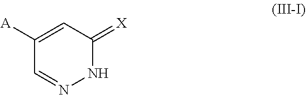

wherein X is S or O and A is as defined herein.

According to a sixth aspect of the invention, there is provided an intermediate compound of formula (III-I)a

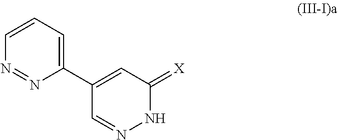

wherein X is S or O.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl, ethyl, n-propyl, 1-methylethyl (iso-propyl), n-butyl, and 1-dimethylethyl (t-butyl).

The process of the present invention can be carried out in separate process steps, wherein the intermediate compounds can be isolated at each stage. Alternatively, the process can be carried out in a one-step procedure wherein the intermediate compounds produced are not isolated. Thus, it is possible for the process of the present invention to be conducted in a batch wise or continuous fashion.

Compounds of formula (I) wherein m is 0 may be represented by a compound of formula (I-Ia) as shown below:

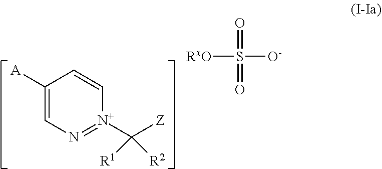

wherein A, R^{x}, R¹, R², and Z are as defined for compounds of formula (I).

Compounds of formula (I) wherein m is 1 may be represented by a compound of formula (I-Ib) as shown below:

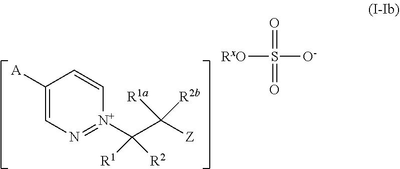

wherein A, R^{x}, R¹, R², R^{1a}, R^{2b} and Z are as defined for compounds of formula (I).

Compounds of formula (I) wherein m is 2 may be represented by a compound of formula (I-Ic) as shown below:

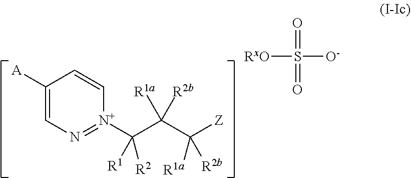

wherein A, R^{x}, R¹, R², R^{1a}, R^{2b} and Z are as defined for compounds of formula (I).

The following list provides definitions, including preferred definitions, for substituents m, p, A, Q, X, Z, R¹, R², R^{1a}, R^{2b}, R³, R⁴, R^{4a}, R^{4b}, R⁵, R^{5a}, R^{5b}, R^{5c}, R^{5d}, R^{5e}, R^{5f}, R^{5g}, R^{5h}, R⁶, R⁷, R⁸, R¹⁰, R^{10a} and R¹² with reference to the compounds and intermediates according to the invention. For any one of these substituents, any of the definitions given below may be combined with any definition of any other substituent given below or elsewhere in this document.

A is a 6-membered heteroaryl selected from the group consisting of formula A-I to A-VII below

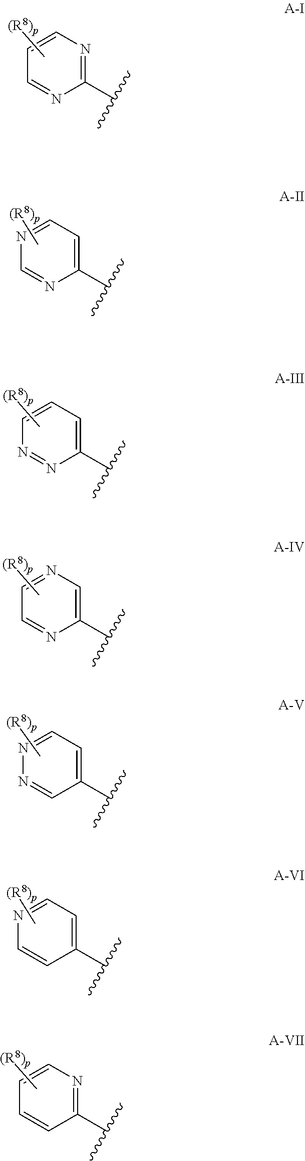

wherein the jagged line defines the point of attachment to the remaining part of a compound of formula (I), p is 0, 1 or 2.

Preferably, A is a 6-membered heteroaryl selected from the group consisting of formula A-I to A-V below

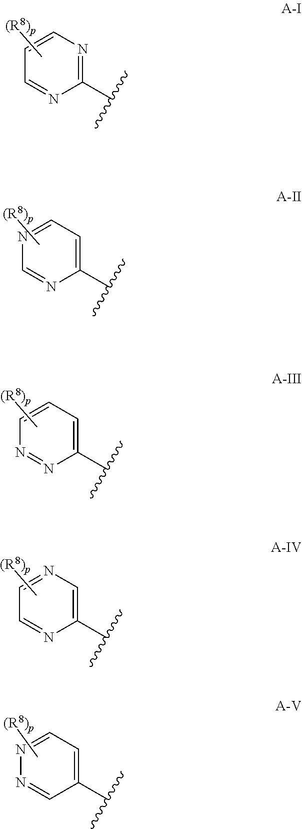

wherein the jagged line defines the point of attachment to the remaining part of a compound of formula (I), p is 0, 1 or 2 (preferably, p is 0 or 1).

More preferably, A is a 6-membered heteroaryl selected from the group consisting of formula A-Ia to A-Va below

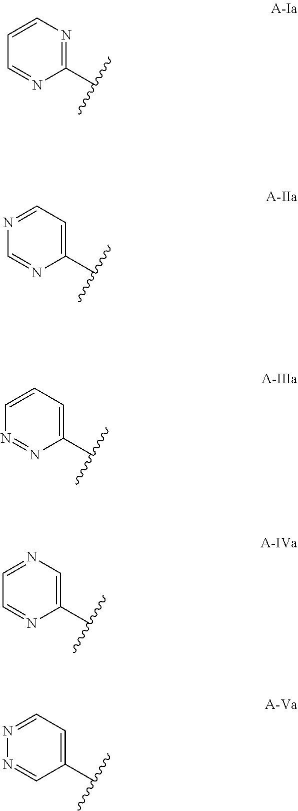 [SMILES: *c1ncccn1 ; *c1ccncn1 ; *c1cccnn1 ; *c1cnccn1 ; *c1ccnnc1]

wherein the jagged line defines the point of attachment to the remaining part of a compound of formula (I).

Even more preferably, A is a 6-membered heteroaryl selected from the group consisting of formula A-Ia to A-IIIa below

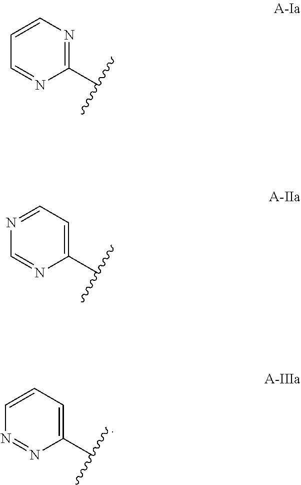 [SMILES: *c1ncccn1 ; *c1ccncn1 ; *c1cccnn1]

wherein the jagged line defines the point of attachment to the remaining part of a compound of formula (I).

Most preferably, A is the group A-Ia or A-IIIa.

In one embodiment, A is the group A-I or A-III below

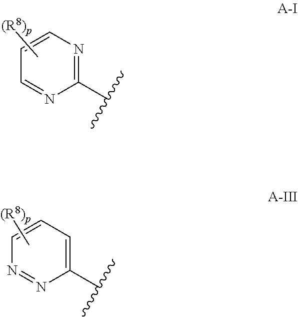

wherein the jagged line defines the point of attachment to the remaining part of a compound of formula (I) and p is 0, 1 or 2 (preferably, p is 0 or 1).

R^{x} is hydrogen or C₁-C₆alkyl. Preferably, R^{x} is selected from the group consisting of hydrogen, methyl, ethyl, n-propyl and iso-propyl. More preferably, R^{x} is selected from the group consisting of hydrogen, methyl and ethyl. Most preferably, R^{x} is hydrogen.

R¹ is hydrogen or methyl, preferably R¹ is hydrogen.

R² is hydrogen or methyl, preferably R² is hydrogen.

In a preferred embodiment R¹ and R² are hydrogen.

Q is (CR^{1a}R^{2b})ₘ.

m is 0, 1 or 2, preferably m is 1 or 2. Most preferably, m is 1.

each R^{1a} and R^{2b} are independently selected from the group consisting of hydrogen, methyl, —OH and —NH₂. More preferably, each R^{1a} and R^{2b} are independently selected from the group consisting of hydrogen and methyl. Most preferably R^{1a} and R^{2b} are hydrogen.

Z is selected from the group consisting of —CN, —C(S)OR¹⁰, —C(S)NR⁶R⁷, —C(S)SR¹⁰, —CH₂OR³, —CH(OR⁴)(OR^{4a}), —C(OR⁴)(OR^{4a})(OR^{4b}), —C(O)OR¹⁰, —C(O)NHCN, —C(O)NR⁶R⁷, —C(O)NHS(O)₂R¹² and —S(O)₂OR¹⁰. Preferably, Z is selected from the group consisting of —CN, —C(S)OR¹⁰, —CH₂OR³, —C(O)OR¹⁰, —C(O)NHCN, —C(O)NR⁶R⁷, —C(O)NHS(O)₂R¹² and —S(O)₂OR¹⁰. More preferably, Z is selected from the group consisting of —CN, —C(O)OR¹⁰, —C(O)NHCN, —C(O)NH₂, —C(O)NHS(O)₂R¹² and —S(O)₂OR¹⁰. Even more preferably, Z is selected from the group consisting of —CN, —C(O)OR¹⁰, —C(O)NH₂ and —S(O)₂OR¹⁰. Yet even more preferably, Z is selected from the group consisting of —CN, —C(O)OCH₂CH₃, —C(O)OC(CH₃)₃, —C(O)OH, —C(O)NH₂ and —S(O)₂OH. Even more preferably still, Z is selected from the group consisting of —CN, —C(O)OCH₂CH₃, —C(O)OC(CH₃)₃, —C(O)OH and —C(O)NH₂.

In an alternative embodiment Z is selected from the group consisting of a group of formula Zₐ, Z_{b}, Z_{c}, Z_{d}, Zₑ and Z_{f} below

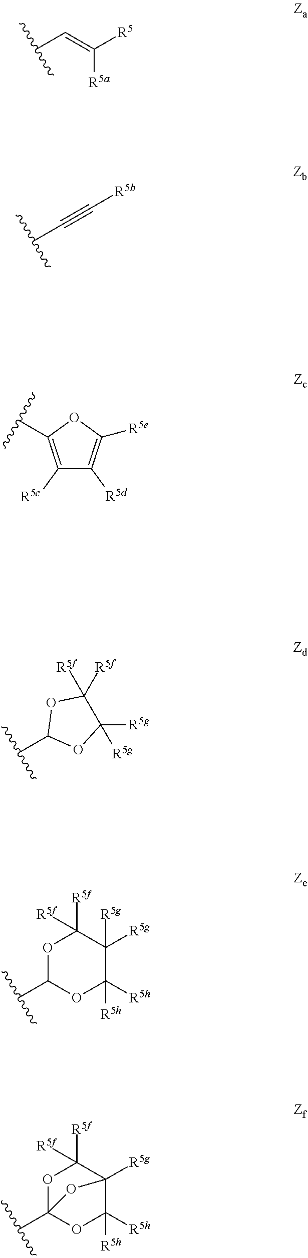

wherein the jagged line defines the point of attachment to the remaining part of a compound of formula (I). Preferably, Z is selected from the group consisting of a group of formula Zₐ, Z_{b}, Z_{d}, Zₑ and Z_{f}. More preferably, Z is selected from the group consisting of a group of formula Zₐ, Z_{d} and Zₑ.

In another embodiment of the invention Z is selected from the group consisting of —CN, —C(O)OCH₂CH₃, —C(O)OC(CH₃)₃ and —C(O)NH₂.

In a further embodiment of the invention Z is selected from the group consisting of —CN, —C(O)OR¹⁰ and —C(O)NH₂ (Preferably, Z is —C(O)OR¹⁰) and R¹⁰ is hydrogen or C₁-C₆alkyl.

The skilled person would appreciate that in specific embodiments R¹⁰ is as defined in specific combination with Z¹ below and that Z¹ and Z² below are subsets of Z for specific embodiments of the invention.

Z¹ is selected from the group consisting of —CN, —C(O)OR¹⁰, —C(O)NH₂ and —S(O)₂OR¹⁰, and R¹⁰ is selected from the group consisting of C₁-C₆alkyl, phenyl and benzyl. Preferably, Z¹ is selected from the group consisting of —CN, —C(O)OR¹⁰ and —C(O)NH₂ and R¹⁰ is C₁-C₆alkyl.

Z² is —C(O)OH or —S(O)₂OH. Preferably, Z² is —C(O)OH.

R³ is hydrogen or —C(O)OR^{10a}. Preferably, R³ is hydrogen.

Each R⁴, R^{4a} and R^{4b} are independently selected from C₁-C₆alkyl. Preferably, each R⁴, R^{4a} and R^{4b} are methyl.

Each R⁵, R^{5a}, R^{5b}, R^{5c}, R^{5d}, R^{5e}, R^{5f}, R^{5g} and R^{5h} are independently selected from hydrogen and C₁-C₆alkyl. More preferably, each R⁵, R^{5a}, R^{5b}, R^{5c}, R^{5d}, R^{5e}, R^{5f}, R^{5g} and R^{5h} are independently selected from hydrogen and methyl. Most preferably, each R⁵, R^{5a}, R^{5b}, R^{5c}, R^{5d}, R^{5e}, R^{5f}, R^{5g} and R^{5h} are hydrogen.

Each R⁶ and R⁷ are independently selected from hydrogen and C₁-C₆alkyl. Preferably, each R⁶ and R⁷ are independently hydrogen or methyl. Most preferably, each R⁶ and R⁷ are hydrogen.

Each R⁸ is independently selected from the group consisting of halo, —NH₂, methyl and methoxy. Preferably, each R⁸ is independently halo (preferably, chloro or bromo) or methyl. More preferably, R⁸ is methyl.

R¹⁰ is selected from the group consisting of hydrogen, C₁-C₆alkyl, phenyl and benzyl. Preferably, R¹⁰ is hydrogen or C₁-C₆alkyl. More preferably, R¹⁰ is selected from the group consisting of hydrogen, methyl, ethyl, iso-propyl, 2,2-dimethylpropyl and tert-butyl. Even more preferably, R¹⁰ is hydrogen, ethyl or tert-butyl.

In one embodiment of the invention, R¹⁰ is ethyl or tert-butyl.

R^{10a} is selected from the group consisting of hydrogen, C₁-C₆alkyl, phenyl and benzyl. Preferably, R^{10a} is selected from the group consisting of hydrogen, C₁-C₆alkyl and phenyl. More preferably, R^{10a} is hydrogen or C₁-C₆alkyl.

R¹² is selected from the group consisting of methyl, —NH₂, —N(CH₃)₂ and —NHCH₃. Preferably, R¹² is methyl.

X is S (sulfur) or O (oxygen).

In one embodiment X is S.

In another embodiment X is O.

Preferably, the compound of formula (I) is further subjected to a salt exchange to give a compound of formula (Id)

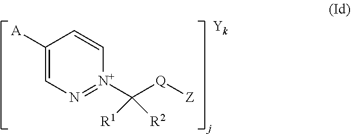

wherein Y represents an agronomically acceptable anion and j and k represent integers that may be selected from 1, 2 or 3, and A, R¹, R², Q and Z are as defined herein.

In another preferred embodiment, there is provided a process for the preparation of a compound of formula (Ie),

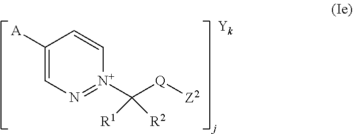

wherein A, R¹, R² and Q are as defined herein and Z² is —C(O)OH or —S(O)₂OH (preferably Z² is —C(O)OH);

comprising:

reacting a compound of formula (II)a:

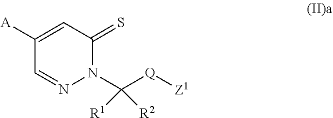

wherein A, R¹, R² and Q are as defined above, and Z¹ is selected from the group consisting of —CN, —C(O)OR¹⁰, —C(O)NH₂ and —S(O)₂OR¹⁰ (preferably, Z¹ is selected from the group consisting of —CN, —C(O)OR¹⁰ and —C(O)NH₂), and R¹⁰ is selected from the group consisting of C₁-C₆alkyl, phenyl and benzyl (preferably, R¹⁰ is C₁-C₆alkyl);

in a suitable reaction medium comprising a desulfurization agent, to give a compound of formula (Ib);

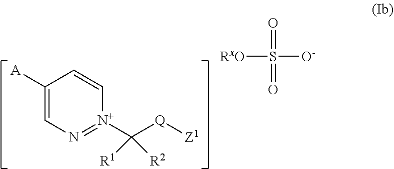

wherein A, R^{x}, R¹, R² and Q are as defined above, and Z¹ is selected from the group consisting of —CN, —C(O)OR¹⁰, —C(O)NH₂ and —S(O)₂OR¹⁰ (preferably, Z¹ is selected from the group consisting of —CN, —C(O)OR¹⁰ and —C(O)NH₂), and R¹⁰ is selected from the group consisting of C₁-C₆alkyl, phenyl and benzyl (preferably, R¹⁰ is C₁-C₆alkyl);

and further subjecting the compound of formula (Ib) to a salt exchange to give a compound of formula (Id-I),

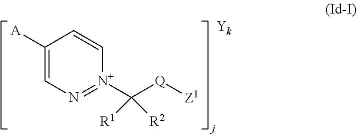

wherein Y, j, k, A, R¹, R² and Q are as defined herein; and

Z¹ is selected from the group consisting of —CN, —C(O)OR¹⁰, —C(O)NH₂ and —S(O)₂OR¹⁰ (preferably, Z¹ is selected from the group consisting of —CN, —C(O)OR¹⁰ and —C(O)NH₂), and R¹⁰ is selected from the group consisting of C₁-C₆alkyl, phenyl and benzyl (preferably, R¹⁰ is C₁-C₆alkyl);

and hydrolysing said compound of formula (Id-I) to a compound of formula (Ie),

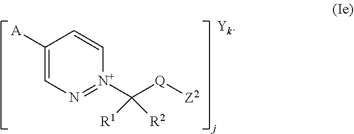

In another preferred embodiment of the invention there is provided a process for the preparation of a compound of formula (Ic)

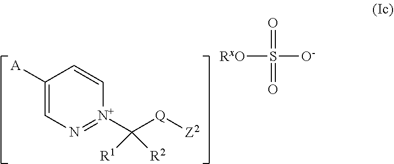

wherein A, R^{x}, R¹, R² and Q are as defined herein and Z² is —C(O)OH or —S(O)₂OH (preferably Z² is —C(O)OH);

said process comprising:

reacting a compound of formula (II)a:

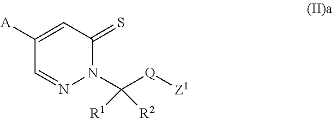

wherein A, R¹, R² and Q are as defined above, and Z¹ is selected from the group consisting of —CN, —C(O)OR¹⁰, —C(O)NH₂ and —S(O)₂OR¹⁰ (preferably, Z¹ is selected from the group consisting of —CN, —C(O)OR¹⁰ and —C(O)NH₂), and R¹⁰ is selected from the group consisting of C₁-C₆alkyl, phenyl and benzyl (preferably, R¹⁰ is C₁-C₆alkyl);

in a suitable reaction medium comprising a desulfurization agent, to give a compound of formula (Ib)

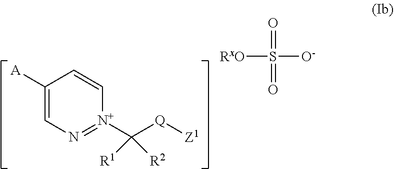

wherein A, R^{x}, R¹, R² and Q are as defined herein, Z¹ is selected from the group consisting of —CN, —C(O)OR¹⁰, —C(O)NH₂ and —S(O)₂OR¹⁰ (preferably, Z¹ is selected from the group consisting of —CN, —C(O)OR¹⁰ and —C(O)NH₂), and R¹⁰ is selected from the group consisting of C₁-C₆alkyl, phenyl and benzyl (preferably, R¹⁰ is C₁-C₆alkyl);

and hydrolysing said compound of formula (Ib) to a compound of formula (Ic),

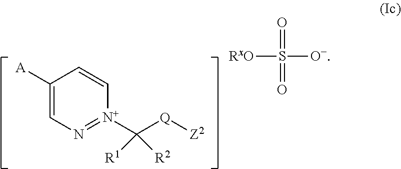

Preferably, the compound of formula (Ic) is further subjected to a salt exchange to give a compound of formula (Ie),

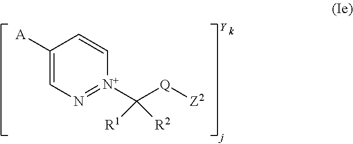

wherein Y represents an agronomically acceptable anion and j and k represent integers that may be selected from 1, 2 or 3, and A, R¹, R² and Q are as defined herein and Z² is —C(O)OH or —S(O)₂OH (preferably Z² is —C(O)OH).

The skilled person would appreciate that compounds of formula (I), (Ib), (Ic), (Id), (Id-I), (Ie) or (Ig), may also exist as a zwitterion (for example, Z is —S(O)₂O⁻) or an agronomically acceptable salt as defined herein. This invention covers processes to make all such agronomically acceptable salts, zwitterions and mixtures thereof in all proportions.

Suitable agronomically acceptable salts in a compound of formula (Id), (Id-I) or (Ie), represented by an anion Y, include but are not limited to chloride, bromide, iodide, fluoride, 2-naphthalenesulfonate, acetate, adipate, aspartate, benzenesulfonate, benzoate, bicarbonate, bisulfate, bitartrate, butylsulfate, butylsulfonate, butyrate, camphorate, camsylate, caprate, caproate, caprylate, carbonate, citrate, diphosphate, edetate, edisylate, enanthate, ethanedisulfonate, ethanesulfonate, ethylsulfate, formate, fumarate, gluceptate, gluconate, glucoronate, glutamate, glycerophosphate, heptadecanoate, hexadecanoate, hydrogen sulfate, hydroxide, hydroxynaphthoate, isethionate, lactate, lactobionate, laurate, malate, maleate, mandelate, mesylate, methanedisulfonate, methylsulfate, mucate, myristate, napsylate, nitrate, nonadecanoate, octadecanoate, oxalate, pelargonate, pentadecanoate, pentafluoropropionate, perchlorate, phosphate, propionate, propylsulfate, propylsulfonate, succinate, sulfate, tartrate, tosylate, tridecylate, triflate, trifluoroacetate, undecylinate and valerate.

Preferably in a compound of formula (Id), (Id-I) or (Ie), Y is chloride, bromide, iodide, hydroxide, bicarbonate, acetate, pentafluoropropionate, triflate, trifluoroacetate, methylsulfate, tosylate and nitrate, and j and k are 1. More preferably, Y is chloride and j and k are 1.

In one embodiment of the invention is provided a compound of formula (I)

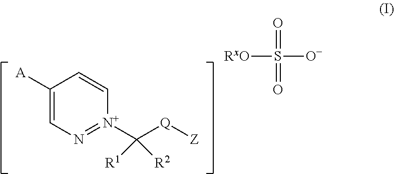

wherein A, R^{x}, R¹, R², Q and Z are as defined herein.

The present invention further provides an intermediate compound of formula (II):

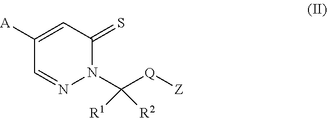

wherein A, R¹, R², Q and Z are as defined herein.

Typically, the compound of formula (II) is produced by:
(i) reacting a compound of formula (III)

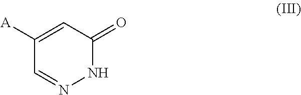

with a suitable alkylating agent (preferably a compound of formula (VI) or (VII)) to give a compound of formula (IV)

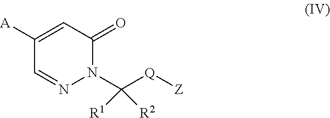

wherein A, R¹, R², Q and Z are as defined herein, and
(ii) reacting the compound of formula (IV) with a sulfurizing agent to give a compound of formula (II)

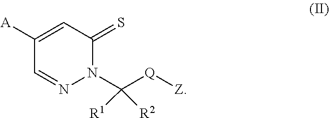

Alternatively, the compound of formula (II) is produced by:
(i) reacting a compound of formula (III)

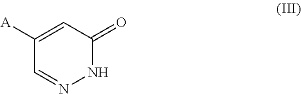

with a sulfurizing agent to give a compound of formula (V),

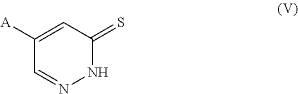

wherein A is as defined herein, and
(ii) reacting the compound of formula (V) with a suitable alkylating (preferably a compound of formula (VI) or (VII)) agent to give a compound of formula (II)

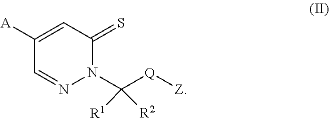

In one embodiment of the invention there is provided the use of a compound of formula (III-I) for preparing a compound of formula (I),

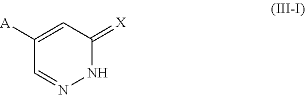

Wherein X is S or O and A is as defined herein (preferably A is A-Ia or A-IIIa).

The present invention still further provides an intermediate compound of formula (III-I)a

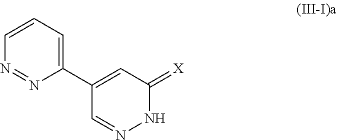

wherein X is S or O.

Compounds of formula (III) are either known in the literature or may be prepared by known literature methods (for example see Alberto Coelho et al. Combinatorial Chemistry & High Throughput Screening, 2006, 9(1), 15-19).

Scheme 1 below describes the reactions of the invention in more detail. The substituent definitions are as defined herein.

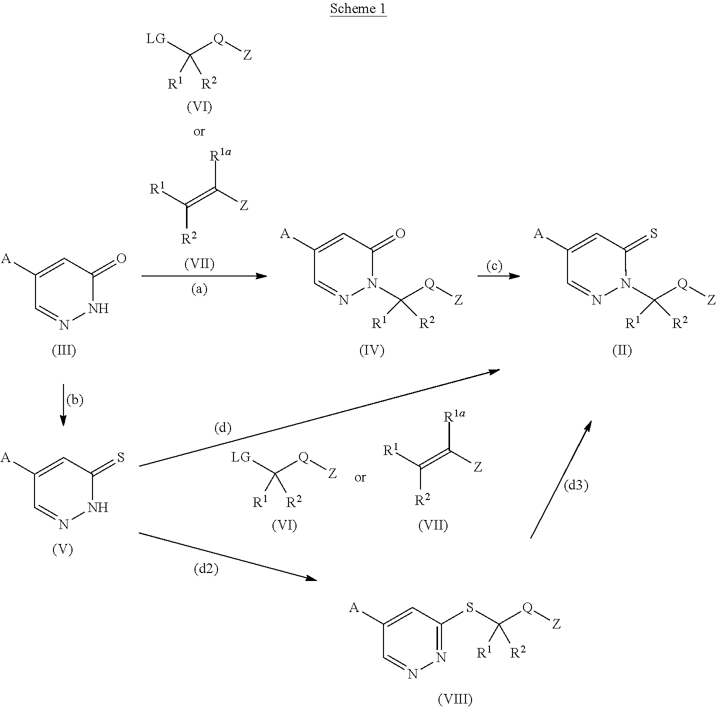

Step (a) Alkylation:

Compounds of formula (IV) can be prepared by reacting a compound of formula (III)

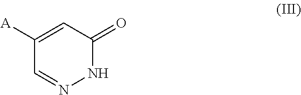

wherein A is as defined herein for the compound of formula (I) with a suitable alkylating agent to give a compound of formula (IV)

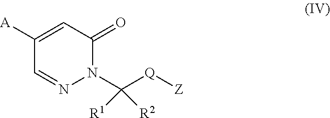

wherein A, R¹, R², Q and Z are as defined herein for compounds of formula (I).

Typically in this process of the invention such suitable alkylating agents may comprise a suitable leaving group (compounds of formula (VI)), for example these may include but are not limited to bromoacetic acid, methyl bromoacetate, 3-bromopropionoic acid, methyl 3-bromopropionate, sodium 2-bromoethanesulphonate, 2,2-dimethylpropyl 2-(trifluoromethylsulfonyloxy)ethanesulfonate, 2-bromo-N-methanesulfonylacetamide, 3-bromo-N-methanesulfonylpropanamide and 3-chloro-2,2-dimethyl-propanoic acid. Alternatively the alkylating agent used in a process of the invention may be a suitably activated electrophilic alkene (compounds of formula (VII), for example these may include but are not limited to acrylic acid, methacrylic acid, acrylonitrile, crotonic acid, 3,3-dimethylacrylic acid, methyl acrylate, ethyl acrylate, tert-butyl acrylate, ethene sulfonic acid, isopropyl ethylenesulfonate and 2,2-dimethylpropyl ethenesulfonate. Alternatively other alkylating agents such as cyclic esters, for example beta-propiolactone or cyclic sulfonic esters, for example gamma-sultone and derivatives thereof are possible.

Preferably, the suitable alkylating agent is either a compound of formula (VI) or formula (VII)

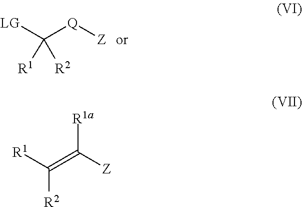

Wherein R¹, R², R^{1a}, Q and Z are as defined herein for compounds of formula (I) and LG is a suitable leaving group (preferably, chloro, bromo or trifluoromethanesulfonate).

More preferably, the suitable alkylating agent is a compound of formula (VII)

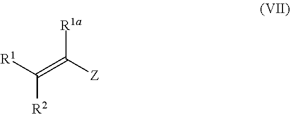

wherein, R¹, R², R^{1a} and Z are as defined above for compounds of formula (I).

In one embodiment, the suitable alkylating agent is selected from the group consisting of beta-propiolactone, acrylonitrile, ethyl acrylate and tert-butyl acrylate. Preferably, the suitable alkylating agent is selected from the group consisting of acrylonitrile, ethyl acrylate and tert-butyl acrylate.

Typically the process described in step (a) is carried out by stirring a compound of formula (III) with an alkylating agent of formula (VI) or (VII) in a solvent, or mixture of solvents, such as acetone, dichloromethane, dichloroethane, N,N-dimethylformamide, acetonitrile, tetrahydrofuran, 2-methyltetrahydrofuran, 1,4-dioxane, water, acetic acid or trifluroacetic acid.

The reaction can be carried out at a temperature of from −78° C. to 150° C., preferably, from 20° C. to 100° C.

The skilled person would appreciate that where required a base can also be used (including, but not limited to, K₂CO₃) and if necessary a phase transfer catalyst (including, but not limited to, tetrabutylammonium bromide).

Preferably process step (a) of the present invention is carried out under an inert atmosphere, such as nitrogen or argon.

Step (b) Sulfurization:

Compounds of formula (V) can be prepared by reacting a compound of formula (III)

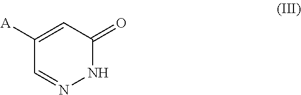

wherein A is as defined above for the compound of formula (I) with a sulfurizing agent to give a compound of formula (V)

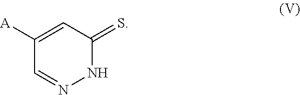

Typically in this process step (b) examples of such sulfurizing agents include but are not limited to, phosphorous pentasulfide (P₂S₅) and lawesson's reagent (2,4-Bis(4-methoxyphenyl)-2,4-dithioxo-1,3,2,4-dithiadiphosphetane). Preferably, the sulfurizing agent is phosphorous pentasulfide.

Typically the process described in step (b) is carried out by stirring a compound of formula (III) with a sulfurizing agent in a solvent, or mixture of solvents, such as chlorobenzene or pyridine.

The reaction can be carried out at a temperature of from 20° C. to 150° C., preferably from 60° C. to 120° C.

Preferably process step (b) of the present invention is carried out under an inert atmosphere, such as nitrogen or argon.

Step (c) Sulfurization:

The compound of formula (II) can be prepared by reacting a compound of formula (IV):

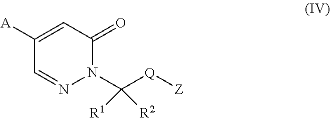

wherein A, R¹, R², Q and Z are as defined herein, with a sulfurizing agent to give a compound of formula (II)

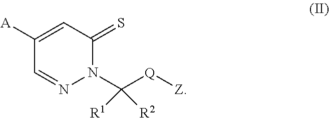

Typically in this process step (c) examples of such sulfurizing agents include but are not limited to, phosphorous pentasulfide (P₂S₅) and lawesson's reagent (2,4-Bis(4-methoxyphenyl)-2,4-dithioxo-1,3,2,4-dithiadiphosphetane). Preferably, the sulfurizing agent is phosphorous pentasulfide.

Typically the process described in step (c) is carried out by stirring a compound of formula (III) with a sulfurizing agent in a solvent, or mixture of solvents, such as chlorobenzene or pyridine.

The reaction can be carried out at a temperature of from 20° C. to 150° C., preferably from 60° C. to 120° C.

Preferably process step (c) of the present invention is carried out under an inert atmosphere, such as nitrogen or argon.

Step (d) Alkylation:

Alternatively, compounds of formula (II) can be prepared by reacting a compound of formula (V)

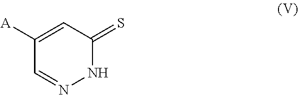

wherein A is as defined above for the compound of formula (I) with a suitable alkylating agent to give a compound of formula (II)

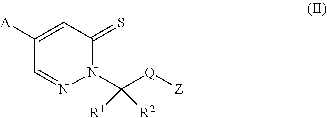

wherein A, R¹, R², Q and Z are as defined above for compounds of formula (I).

Typically in this process of the invention such suitable alkylating agents may comprise a suitable leaving group (compounds of formula (VI)), for example these may include but are not limited to bromoacetic acid, methyl bromoacetate, 3-bromopropionoic acid, methyl 3-bromopropionate, sodium 2-bromoethanesulphonate, 2,2-dimethylpropyl 2-(trifluoromethylsulfonyloxy)ethanesulfonate, 2-bromo-N-methanesulfonylacetamide, 3-bromo-N-methanesulfonylpropanamide and 3-chloro-2,2-dimethyl-propanoic acid. Alternatively the alkylating agent used in a process of the invention may be a suitably activated electrophilic alkene (compounds of formula (VII), for example these may include but are not limited to acrylic acid, methacrylic acid, acrylonitrile, crotonic acid, 3,3-dimethylacrylic acid, methyl acrylate, ethyl acrylate, tert-butyl acrylate, ethene sulfonic acid, isopropyl ethylenesulfonate and 2,2-dimethylpropyl ethenesulfonate. Alternatively, other alkylating agents such as cyclic esters, for example beta-propiolactone or cyclic sulfonic esters, for example gamma-sultone and derivatives thereof are possible.

Preferably, the suitable alkylating agent is either a compound of formula (VI) or formula (VII)

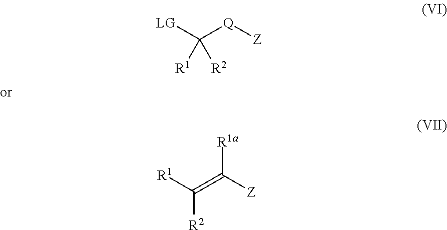

Wherein R¹, R², R^{1a}, Q and Z are as defined above for compounds of formula (I) and LG is a suitable leaving group (preferably, chloro, bromo or trifluoromethanesulfonate).

More preferably, the suitable alkylating agent is a compound of formula (VII)

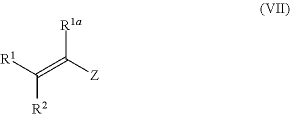

wherein, R¹, R², R^{1a} and Z are as defined above for compounds of formula (I).

In one embodiment, the suitable alkylating agent is selected from the group consisting of beta-propiolactone, acrylonitrile, ethyl acrylate and tert-butyl acrylate. Preferably, the suitable alkylating agent is selected from the group consisting of acrylonitrile, ethyl acrylate and tert-butyl acrylate.

Typically the process described in step (d) is carried out by stirring a compound of formula (V) with an alkylating agent of formula (VI) or (VII) in a solvent, or mixture of solvents, such as acetone, dichloromethane, dichloroethane, N,N-dimethylformamide, acetonitrile, tetrahydrofuran, 2-methyltetrahydrofuran, 1,4-dioxane, water, acetic acid or trifluroacetic acid.

The reaction can be carried out at a temperature of from −78° C. to 150° C., preferably, from 20° C. to 100° C.

The skilled person would appreciate that where required a base can also be used (including, but not limited to, K₂CO₃) and if necessary a phase transfer catalyst (including, but not limited to, tetrabutylammonium bromide).

Preferably process step (d) of the present invention is carried out under an inert atmosphere, such as nitrogen or argon.

Step (d2) and (d)3—Alternative Alkylation

The skilled person would appreciate that the described step (d) Alkylation may proceed via intermediacy of compound of formula (VIII)

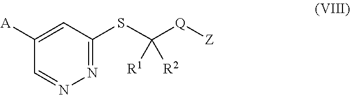

wherein A, R¹, R², Q and Z are as defined above for compounds of formula (I).

Steps (d2) S-Alkylation and (d3) Rearrangement may be carried out in one vessel (one-pot transformation) or sequentially (different reaction vessels).

Typically the process described in step (d3) is carried out in the presence of a base, including, but not limited to, sodium carbonate, potassium carbonate, sodium hydroxide, potassium hydroxide, DBU, tetrabutyl ammonium hydroxide or Amberlite® resin. The amount of the base is typically between 0.01 and 1 equivalent, preferentially between 0.01 and 0.5 equivalent. Additionally, the process can be carried out in presence of a phase transfer catalyst including, but not limited to, tetrabutylammonium bromide or a nucleophilic catalyst including, but not limited to tetrabutyl ammonium iodide and potassium iodide. The amount of the catalyst is typically between 0.01 and 1 equivalent.

The process is typically carried out in a suitable solvent. Suitable solvents thus include but are not limited to, for example acetonitrile, propanenitrile, dimethyl formamide, dimethyl sulphoxide, N-methyl pyrrolidone (NMP), dimethyl acetamide, sulfolane, cyclohexane, n-hexane, methyl cyclohexane, heptane, chlorobenzene, 1,2-dichlorobenzene, methyl acetate, dimethyl carbonate, ethyl acetate, isopropyl acetate, propyl acetate, t-butyl acetate, ethylene carbonate, propylene carbonate, butyl acetate, butyrolactone, butyronitrile, toluene, xylene iso-mix, cumene, isopropylbenzene, p-xylene, mesitylene, benzonitrile, nitrobenzene, o-xylene, m-xylene, ethylbenzene, methanol, iso-Amyl alcohol, isopropanol, t-Butanol and t-amyl alcohol, tetrahydrofuran, 2-methyl tetrahydrofuran, 1,4-dioxane. Step (d3) may be an equilibrium reaction and various methods know to shift the reaction equilibria towards the desired product may be used, including, but not limited to preferential crystallization of the desired product.

Scheme 2 below describes the reactions of the invention in more detail. The substituent definitions are as defined herein.

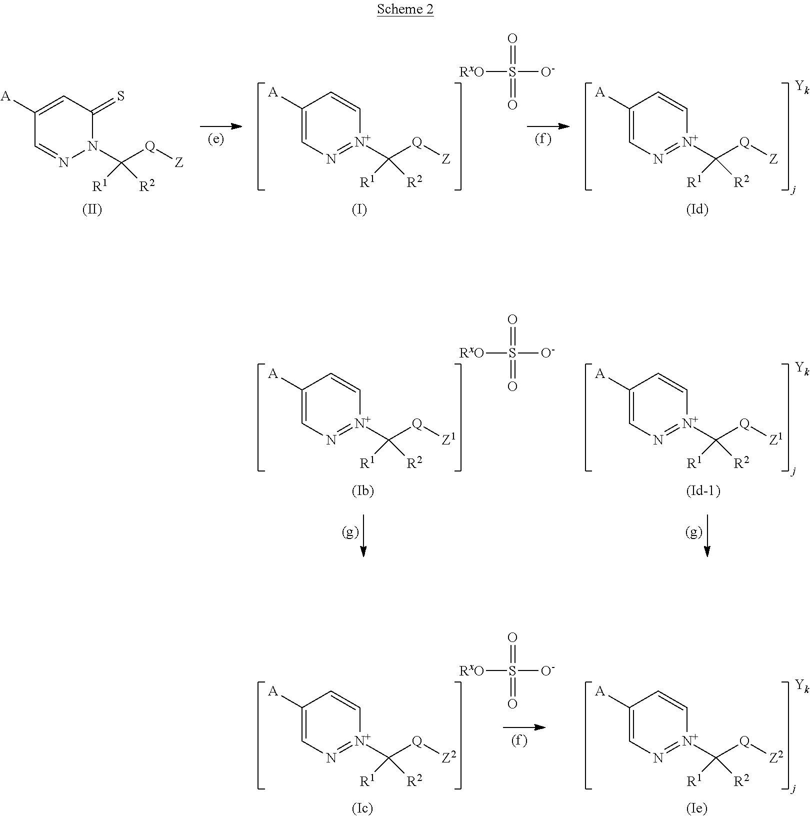

Step (e) Desulfurization:

Compounds of formula (I)

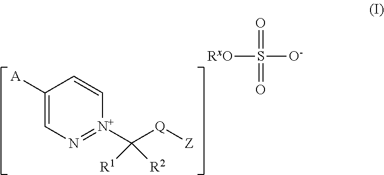

wherein A, R^{x}, R¹, R², Q and Z are as defined above, can be prepared by, reacting a compound of formula (II):

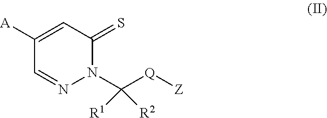

wherein A, R¹, R², Q and Z are as defined above for compound of formula (I), in a suitable reaction medium comprising a desulfurization agent, to give a compound of formula (I).

The process according to the invention is typically carried out in a suitable reaction medium, which can be a solvent which is in principle any solvent or mixture of solvents that are inert under the reaction conditions. The skilled person would appreciate that were for example the desulfurization agent is hydrogen peroxide then this may be provided, for example, as a 27 wt % solution in water which may act as suitable reaction medium.

Suitable solvents thus include but are not limited to, for example, water, acetonitrile, propanenitrile, formamide, dimethyl formamide, N-methylformamide, dimethyl sulphoxide, N-methyl pyrrolidone (NMP), dimethyl acetamide, 1,3-Dimethyl-2-imidazolidinone, sulfolane, N-butylpyrrolidone (NBP), N-octylpyrrolidone, cyclohexane, pentane, 2-methylpentane, n-hexane, isooctane, methyl cyclohexane, heptane, methylcyclopentane, petroleum spirit, cis-decalin, n-octane, nonane, decane, limonene, trifluorotoluene, chlorobenzene, 1,2-dichlorobenzene, 1,2,4-trichlorobenzene, 1,1-dichloroethane, 1,1,1-trichloroethane, trichloroethylene, bromobenzene, 1-chlorobutane, perfluoromethylcyclohexane, iodobenzene, dichloromethane, chloroform, perfluorohexane, 1,2-dichloroethane, perfluorotoluene, perfluorocyclohexane, chloroacetic acid, trichloroacetic acid, propionic acid, acetic acid, acetic anhydride, formic acid, n-butanoic acid, n-pentanoic acid, n-hexanoic acid, propionic anhydride, methyl acetate, dimethyl carbonate, ethyl acetate, ethyl formate, isopropyl acetate, propyl acetate, methyl lactate, ethyl propionate, t-butyl acetate, ethylene carbonate, propylene carbonate, butyl acetate, ethyl lactate, n-octyl acetate, diethyl carbonate, iso-butylacetate, formic acid methyl ester, butyrolactone, methyl benzoate, dimethyl phthalate, ethyl benzoate, i-pentyl acetate, methyl propionate, butyronitrile, N,N-diethylacetamide, tetraethylurea, N,N-diethylpropionamide, valeronitrile, malononitrile, tetramethylurea, N,N-dimethyltrifluoroacetamide, N,N-dimethylchloroacetamide, di-n-butyl sulfoxide, N,N-diethylbenzamide, toluene, xylene iso-mix, cumene, isopropylbenzene, p-xylene, mesitylene, benzonitrile, nitrobenzene, o-xylene, m-xylene, ethylbenzene, tetralin, methanol, iso-Amyl alcohol, isopropanol, t-Butanol and t-amyl alcohol.

In a preferred embodiment of the invention the suitable reaction medium further comprises an acid. Preferably the acid is selected from the group consisting of chloroacetic acid, trichloroacetic acid, propionic acid, acetic acid, acetic anhydride, formic acid, n-butanoic acid, n-pentanoic acid, n-hexanoic acid and propionic anhydride. More preferably, the acid is acetic acid and/or formic acid.

In one embodiment of the invention the suitable reaction medium comprises water and an acid (preferably, formic acid and/or acetic acid).

In another embodiment of the invention the suitable reaction medium comprises ethyl acetate, water and formic acid and/or acetic acid.

Preferably, the desulfurization agent in the process according to the invention is an oxidant. In principle any oxidation reagent known to a person skilled in the art for oxidation of an organic sulfide group could be employed.

Suitable oxidizing agents include, but are not limited to, hydrogen peroxide, hydrogen peroxide and a suitable catalyst (for example, but are not limited to: TiCl₃, Mn(OAc)₃.2H₂O and a bipyridine ligand, VO(acac)₂ and a bidentate ligand, Ti(OiPr₄) and a bidentate ligand, Polyoxymetalates, Na₂WO₄ together with additives such as PhPO₃H₂ and CH₃(n-C₈H₁₇)₃NHSO₄, lanthanide catalysts such as Sc(OTf)₃, organic molecules can also act as catalysts, for example flavins), chlorine, with or without a suitable catalyst (as listed above), bromine with or without a suitable catalyst (as listed above), organic hydroperoxides (for example peracetic acid, performic acid, t-Butylhydroperoxide, cumylhydroperoxide, m-CPBA (meta-Chloroperoxybenzoic acid)), an organic hydroperoxide prepared in situ (for example from the reaction of H₂O₂ and a carboxylic acid+a suitable catalyst), organic peroxides (for example benzoyl peroxide, or di-terbutylperoxide), amine N-oxides (for example N-Methylmorpholine Oxide, pyridine N-oxide or triethylamine N-oxide peroxide derivative), inorganic oxidants (NalO₄, KMnO₄, MnO₂, CrO₃), inorganic oxidants prepared in situ (for example, a Ru catalyst+an oxidant forms in situ RuO4 which maybe a capable oxidant), inorganic hydroperoxides, inorganic peroxides, dioxiranes (for example DMDO), oxone, ozone, oxygen (oxygen+a suitable catalyst such as NO₂, or Cerric ammonium nitrate), air+a suitable catalyst (such systems can lead to the in-situ formation of peroxides and suitable catalysts can be for example, but not limited to, Fe(NO₃)₃—FeBr₃), NaOCl (which may be used in conjunction with catalytic amounts of a stable radical such as (2,2,6,6-tetramethylpiperidin-1-yl)oxyl (TEMPO), 4-hydroxy-TEMPO or 4-acetylamino-TEMPO, optionally catalytic amounts of sodium bromide may also be added), NaOBr, HNO₃, biocatalysts such as peroxidases and monooxygenases and nitrosyl chloride (prepared in situ).

Preferably, the desulfurization agent is a peroxide or derivative thereof (for example peracetic acid, performic acid, t-Butylhydroperoxide, cumylhydroperoxide, m-CPBA). Most preferably, the desulfurization agent is hydrogen peroxide.

The skilled person would appreciate that the temperature of the process according to the invention can vary depending on the choice of solvent used. Typically, the process according to the invention is carried out at a temperature from 40° C. to 120° C., preferably from 80° C. to 110° C.

Preferably, the process of the present invention is carried out under an inert atmosphere, such as nitrogen or argon.

Step (f) Salt Exchange:

Compounds of formula (Id),

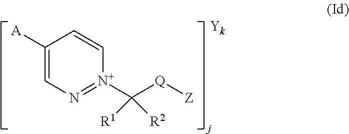

wherein Y represents an agronomically acceptable anion and j and k represent integers that may be selected from 1, 2 or 3, and A, R¹, R², Q and Z are as defined herein, may be prepared by a salt exchange of a compound of formula (I),

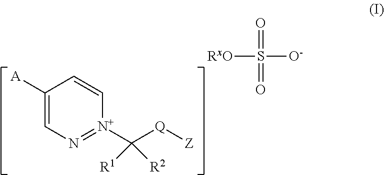

wherein A, R^{x}, R¹, R², Q and Z are as defined herein.

Likewise, compounds of formula (Ie),

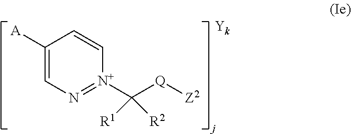

wherein Y represents an agronomically acceptable anion and j and k represent integers that may be selected from 1, 2 or 3, and A, R¹, R² and Q are as defined herein and Z² is —C(O)OH or —S(O)₂OH (preferably Z² is —C(O)OH), may be prepared by a salt exchange of a compound of formula (Ic),

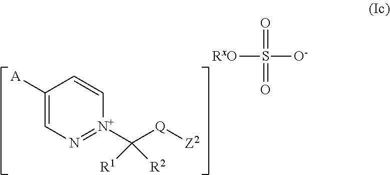

wherein A, R^{x}, R¹, R² and Q are as defined in claim 1 and Z² is —C(O)OH or —S(O)₂OH (preferably Z² is —C(O)OH).

The salt exchange of a compound of formula (I) to a compound of formula (Id) or a compound of formula (Ic) to a compound of formula (Ie) can be performed using methods known to a person skilled in the art and refers to the process of converting one salt form of a compound into another, for example converting a hydrogen sulfate (HSO₄⁻) salt to a chloride (Cl⁻) salt. The salt exchange is typically performed using an ion exchange resin or a water soluble salt, for example, Amberlite® resin (preferably a strong base anion exchange resin) or barium chloride (BaCl₂). Preferably, the salt exchange of a compound of formula (I) to a compound of formula (Id) or a compound of formula (Ic) to a compound of formula (Ie) is performed with barium chloride.

Step (g) Hydrolysis:

Compounds of formula (Ic)

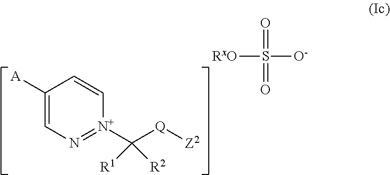

wherein A, R^{x}, R¹, R² and Q are as defined herein and Z² is —C(O)OH or —S(O)₂OH (preferably Z² is —C(O)OH) can be prepared by hydrolyzing a compound of formula (Ib)

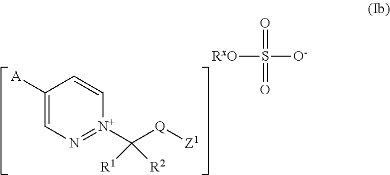

wherein A, R^{x}, R¹, R² and Q are as defined herein, Z¹ is selected from the group consisting of —CN, —C(O)OR¹⁰, —C(O)NH₂ and —S(O)₂OR¹⁰ (preferably, Z¹ is selected from the group consisting of —CN, —C(O)OR¹⁰ and —C(O)NH₂), and R¹⁰ is selected from the group consisting of C₁-C₆alkyl, phenyl and benzyl (preferably, R¹⁰ is C₁-C₆alkyl).

Likewise, compounds of formula (Ie)

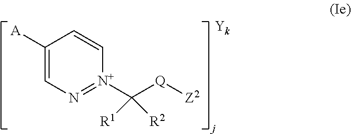

wherein Y represents an agronomically acceptable anion and j and k represent integers that may be selected from 1, 2 or 3, and A, R¹, R² and Q are as defined herein and Z² is —C(O)OH or —S(O)₂OH (preferably Z² is —C(O)OH) can be prepared by hydrolyzing a compound of formula (Id-I),

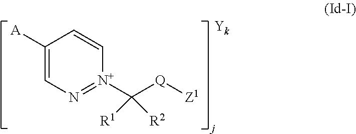

wherein A, R¹, R² and Q are as defined herein, Z¹ is selected from the group consisting of —CN, —C(O)OR¹⁰, —C(O)NH₂ and —S(O)₂OR¹⁰ (preferably, Z¹ is selected from the group consisting of —CN, —C(O)OR¹⁰ and —C(O)NH₂), and R¹⁰ is selected from the group consisting of C₁-C₆alkyl, phenyl and benzyl (preferably, R¹⁰ is C₁-C₆alkyl).

The hydrolysis can be performed using methods known to a person skilled in the art. The hydrolysis is typically performed using a suitable reagent, including, but not limited to aqueous sulfuric acid, concentrated hydrochloric acid or an acidic ion exchange resin.

Typically, the hydrolysis is carried out using aqueous hydrochloric acid (for example but not limited to, 32 wt % aq. HCl) or a mixture of HCl and an appropriate solvent, (such as but not limited to acetic acid, isobutyric acid or propionic acid), optionally in the presence of an additional suitable solvent (for example, but not limited to, water), at a suitable temperature from 0° C. to 120° C. (preferably, from 20° C. to 100° C.).

In a preferred embodiment of the invention there is provided a process for the preparation of a compound of formula (Ig)

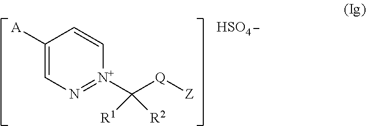

wherein

A is a 6-membered heteroaryl selected from the group consisting of formula A-Ia to A-IIIa below

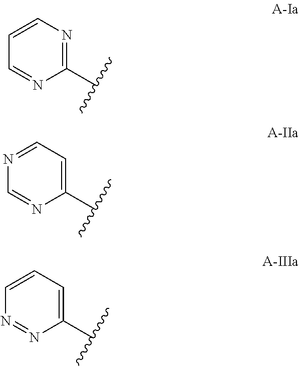 [SMILES: *c1ncccn1 ; *c1ccncn1 ; *c1cccnn1]

wherein the jagged line defines the point of attachment to the remaining part of a compound of formula (I); and

R¹ is hydrogen;

R² is hydrogen;

Q is (CR^{1a}R^{2b})ₘ;

m is 1;

each R^{1a} and R^{2b} are hydrogen;

Z is selected from the group consisting of —CN, —C(O)OR¹⁰, —C(O)NH₂ and —S(O)₂OR¹⁰ (preferably, Z is selected from the group consisting of —CN, —C(O)OR¹⁰ and —C(O)NH₂); and

R¹⁰ is selected from the group consisting of hydrogen and C₁-C₆alkyl;

said process comprising:

reacting a compound of formula (II):

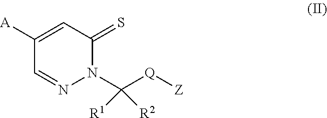

wherein A, R¹, R², Q and Z are as defined above, in a suitable reaction medium comprising an oxidant (preferably a peroxide or derivative thereof, more preferably a peroxide selected from the list consisting of hydrogen peroxide, peracetic acid, performic acid, t-Butylhydroperoxide, cumylhydroperoxide and m-CPBA) and an acid (preferably, the acid is selected from the group consisting of chloroacetic acid, trichloroacetic acid, propionic acid, acetic acid, acetic anhydride, formic acid, n-butanoic acid, n-pentanoic acid, n-hexanoic acid and propionic anhydride), to give a compound of formula (Ig).

In a more preferred embodiment of the invention there is provided a process for the preparation of a compound of formula (Ig)

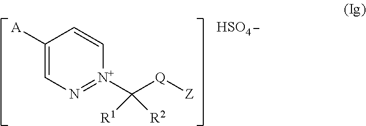

wherein

A is a 6-membered heteroaryl selected from the group consisting of formula A-Ia to A-IIIa below

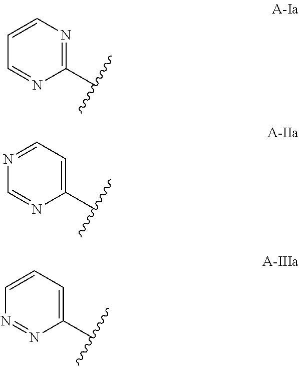 [SMILES: *c1ncccn1 ; *c1ccncn1 ; *c1cccnn1]

wherein the jagged line defines the point of attachment to the remaining part of a compound of formula (I); and

R¹ is hydrogen;

R² is hydrogen;

Q is (CR^{1a}R^{2b})ₘ;

m is 1;

each R^{1a} and R^{2b} are hydrogen;

Z is selected from the group consisting of —CN, —C(O)OR¹⁰, —C(O)NH₂ and —S(O)₂OR¹⁰ (preferably, Z is selected from the group consisting of —CN, —C(O)OR¹⁰ and —C(O)NH₂); and

R¹⁰ is selected from the group consisting of hydrogen and C₁-C₆alkyl;

said process comprising:

reacting a compound of formula (II):

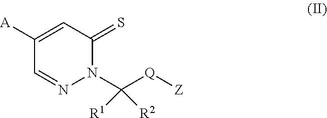

wherein A, R¹, R², Q and Z are as defined above, in a suitable reaction medium comprising acetic acid and/or formic acid and hydrogen peroxide, to give a compound of formula (Ig).

### EXAMPLES

The following examples further illustrate, but do not limit, the invention. Those skilled in the art will promptly recognise appropriate variations from the procedures both as to reactants and as to reaction conditions and techniques.

The following abbreviations are used: s=singlet; br s=broad singlet; d=doublet; dd=double doublet; dt=double triplet; t=triplet, tt=triple triplet, q=quartet, quin=quintuplet, sept=septet; m=multiplet; GC=gas chromatography, RT=retention time, Tᵢ=internal temperature, MH⁺=molecular mass of the molecular cation, M=molar, Q¹HNMR=quantitative ¹HNMR, RT=room temperature, TBME=tert-butyl methyl ether, UFLC=Ultra-fast liquid chromatography.

UFLC (UPLC) Methods:

Standard:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85

Standard Long:

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 2.7 min; Flow (ml/min) 0.85

Standard Long Polar:

Spectra were recorded on a Mass Spectrometer from Waters (SQD, SQDII or ZQ Single quadrupole mass spectrometer) equipped with an electrospray source (Polarity: positive and negative ions), Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 0-10% B in 2.5 min; Flow (ml/min) 0.85

Apolar:

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 40-100% B in 1.2 min; Flow (ml/min) 0.85

¹H NMR spectra are recorded at 400 MHz unless indicated otherwise and chemical shifts are recorded in ppm.

### Preparation of Ethyl 3-(6-oxo-4-pyrimidin-2-yl-pyridazin-1-yl) propanoate (5A)

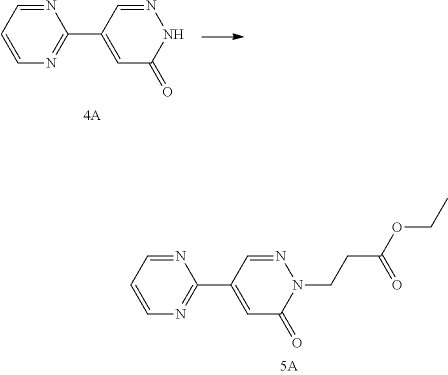 [SMILES: O=c1cc(-c2ncccn2)cn[nH]1 ; CCOC(=O)CCn1ncc(-c2ncccn2)cc1=O]

General Procedure 1—Alkylation:

To a three neck round bottom flask (250 mL), charge 2-methyltetrahydrofuran (50 mL) at 24° C. under N₂ atmosphere. Start stirring at 300-400 rpm. Charge 4-pyrimidin-2-yl-1H-pyridazin-6-one (5.00 g, 25.40 mmol). Charge K₂CO₃ (1.40 g, 0.40 eq., 10.20 mmol) followed by tetrabutylammonium bromide (0.42 g, 0.05 eq., 1.27 mmol, 98.00 mass %). Heat the reaction mixture to 80° C. Charge ethyl prop-2-enoate (7.71 g, 3.00 eq., 76.20 mmol) dropwise with syringe pump over a period of 15 min. Continue the reaction at 80° C. for 60 min with monitoring the progress on UFLC. Cool the reaction to 24° C., add water (50 mL) and stir for 20 min. Evaporate the volatile solvents under vacuum at 45-50° C. Charge water (50 mL), stir for 15 min, filter and dry under vacuum to give ethyl 3-(6-oxo-4-pyrimidin-2-yl-pyridazin-1-yl) propanoate (5A) (6.70 g, 88% yield, 91.5% assay).

1H NMR (400 MHz, DMSO-d6) δ ppm 1.15 (t, J=7.09 Hz, 3H) 2.81 (t, J=6.97 Hz, 2H) 4.05 (q, J=7.09 Hz, 2H) 4.34 (t, J=6.91 Hz, 2H) 7.60-7.65 (m, 2H) 8.65 (d, J=2.08 Hz, 1H) 9.00 (d, J=4.89 Hz, 2H)

### Preparation of Ethyl 3-(4-pyrimidin-2-yl-6-thioxo-pyridazin-1-yl)propanoate (6A)

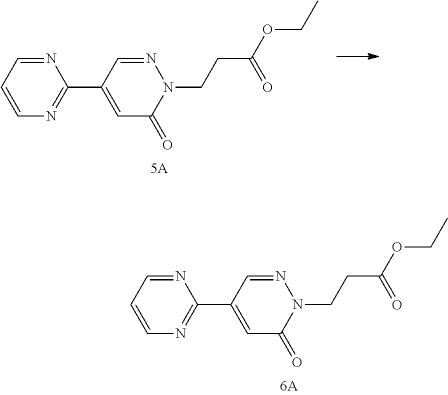 [SMILES: CCOC(=O)CCn1ncc(-c2ncccn2)cc1=O ; CCOC(=O)CCn1ncc(-c2ncccn2)cc1=O]

General Procedure 2—Sulfurization:

To a three neck round bottom flask (250 mL), charge Chlorobenzene (150 mL) at 24° C. under N₂ atmosphere. Start stirring at 300-400 rpm. Charge P₂S₅ (5.25 g, 23.38 mmol, 0.45 eq.), N,N-diethylaniline (3.48 g, 23.38 mmol, 0.45 eq.). Heat to 100° C. Charge ethyl 3-(6-oxo-4-pyrimidin-2-yl-pyridazin-1-yl)propanoate (1) (15.00 g, 51.95 mmol, 1.00 eq.) portionwise over 60 min. Stir the reaction at 100° C. for 120 min and monitor the progress on UFLC. Cool the reaction mixture to 24° C. and filter through Celite® bed. Wash the filtrate with water (60 mL) and separate layers. To the chlorobenzene layer, charge water (60 mL) and adjust pH=12 with 2-5% aq. NaOH solution. Separate the layers and wash chlorobenzene layer with water (45 mL) and brine (25 mL). Separate layers. Distill out ˜90% of chlorobenzene from chlorobenzene layer under reduced pressure (100-150 mbar) at 65° C. Add methylcyclohexane (292 mL) at 65° C. and stir for 10-15 min. Cool the reaction mixture and stir at 0° C. for 60 min. Filter the desired product ethyl 3-(4-pyrimidin-2-yl-6-thioxo-pyridazin-1-yl)propanoate (6A) as orange color solid (13.64 g, 85% yield, 94.0% assay).

6A: ¹H NMR (400 MHz, DMSO-d₆) δ ppm 1.12-1.21 (m, 3H) 2.93-3.01 (m, 2H) 4.03-4.15 (m, 2H) 4.74-4.85 (m, 2H) 7.62-7.70 (m, 1H) 8.38-8.46 (m, 1H) 8.97-9.10 (m, 3H)

### Preparation of Ethyl 3-(4-pyrimidin-2-ylpyridazin-1-ium-1-yl)propanoate hydrogen sulfate (7A)

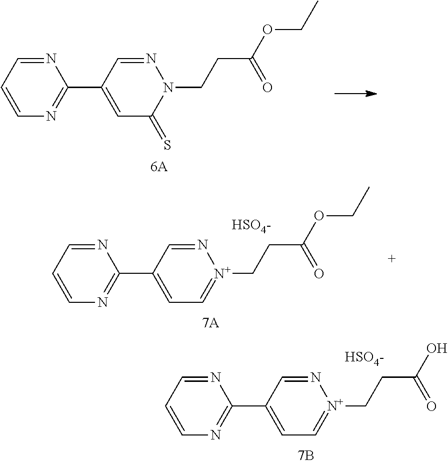

General Procedure 3—Desulfurization, Formic Acid-H₂O₂ as Oxidant

To a three neck round bottom flask (250 mL), charge ethyl acetate (40 mL), water (20 mL) and formic acid (10 mL) at 24° C. under N₂ atmosphere. Start stirring at 300-400 rpm. Charge ethyl 3-(4-pyrimidin-2-yl-6-thioxo-pyridazin-1-yl)propanoate (6A) (10.00 g, 33.72 mmol, 1.00 eq., 97.79 mass %) at 24° C. and stir for 10 min. Dose H₂O₂ (11.50 mL, 101.20 mmol, 3.00 eq., 27% in H₂O) over the period of 180 min. Stir the reaction for 180 min and monitor the progress on UFLC. Charge water (80 mL), stir for 15 min and separate the layers. Extract the aqueous layer with ethyl acetate (3×30 mL) and separate the layers. Quench the unreacted H₂O₂ in aqueous layer with charcoal (1 g) and stir for 15 h at 24° C. Filter through Celite® bed to give clear aqueous layer (103.86 g). The aqueous layer was analysed by quantitative 1HNMR using an internal standard and was composed of ethyl 3-(4-pyrimidin-2-ylpyridazin-1-ium-1-yl)propanoate hydrogen sulfate (7A) (7.73% w/w, 67% yield) and 3-(4-pyrimidin-2-ylpyridazin-1-ium-1-yl)propanoic acid hydrogen sulfate (7B) (0.71% w/w, 6.7% yield).

7A: ¹H NMR (400 MHz, D₂O) δ ppm: 10.12-10.13 (m, 1H), 9.92-9.90 (m, 1H), 9.36-9.34 (m, 1H), 9.15-9.11 (m, 1H), 8.57-8.54 (m, 1H), 8.06-8.02 (m, 1H), 5.18-5.13 (m, 2H), 4.07 (q, J=8.0 Hz, 2H), 3.27 (t, J=8.0 Hz, 2H), 1.12 (t, J=8.0 Hz, 3H).

General Procedure 4—Desulfurization, Acetic Acid-H₂O₂ as Oxidant

To a solution of ethyl 3-(4-pyrimidin-2-yl-6-thioxo-pyridazin-1-yl)propanoate (6A) (3 g, 10.13 mmol, 1 eq.) in acetic acid (68 ml) was added slowly hydrogen peroxide (30% w/w in H₂O, 33.42 mmol, 3.3 eq.). The reaction was stirred at room temperature for 2 hours, before solid sodium metabisulfite (5.07 mmol) was added to the mixture. The reaction mixture was concentrated under vacuum to yield the title compound 7A as an orange solid (5.5 g, 59% assay, 90% yield).

7A: ¹H NMR (400 MHz, D₂O) δ ppm: 10.12-10.13 (m, 1H), 9.92-9.90 (m, 1H), 9.36-9.34 (m, 1H), 9.15-9.11 (m, 1H), 8.57-8.54 (m, 1H), 8.06-8.02 (m, 1H), 5.18-5.13 (m, 2H), 4.07 (q, J=8.0 Hz, 2H), 3.27 (t, J=8.0 Hz, 2H), 1.12 (t, J=8.0 Hz, 3H).

### Preparation of 3-(4-pyrimidin-2-ylpyridazin-1-ium-1-yl)propanoic acid hydrogen sulfate (7B)

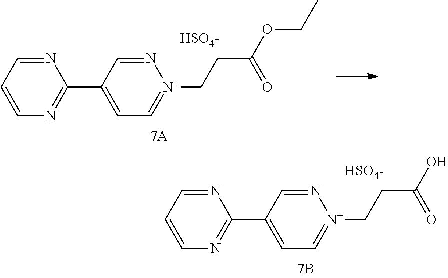 [SMILES: CCOC(=O)CC[n+]1ccc(-c2ncccn2)cn1 ; "HSO4-" ; O=C(O)CC[n+]1ccc(-c2ncccn2)cn1 ; "HSO4-"]

General Procedure 5—Sulfuric Acid Hydrolysis

To a three neck round bottom flask (250 mL), charge aqueous layer containing ethyl 3-(4-pyrimidin-2-ylpyridazin-1-ium-1-yl)propanoate hydrogen sulfate (7A), and 3-(4-pyrimidin-2-ylpyridazin-1-ium-1-yl)propanoic acid hydrogen sulfate (7B) (103.10 g, 68% w/w of 7A and 7.8% w/w of 7B) at 24° C. under N₂ atmosphere. Start stirring at 300-400 rpm. Charge sulfuric acid (50.00 mg, 0.51 mmol, cat.). Heat the reaction mass to 95° C. for 240 min and monitor the hydrolysis on UFLC. Cool the reaction mass to 24° C. and evaporate to dryness to give desired product (11.60 g, 79% yield over 2 steps, 75.6% assay). Crystallization in water/isopropanol/acetone (1:2:2) (67 mL), followed by filtration gives 3-(4-pyrimidin-2-ylpyridazin-1-ium-1-yl)propanoic acid hydrogen sulfate (7B) as an off-white solid (8.54 g, 70% from 6A, 91.3% assay).

7B: ¹H NMR (400 MHz, D₂O) δ ppm: 3.32 (t, J=6.11 Hz, 2H), 5.18 (t, J=6.11 Hz, 2H), 7.71 (t, J=5.00 Hz, 1H), 9.06 (d, J=5.08 Hz, 2H), 9.25 (dd, J=6.19, 2.38 Hz, 1H), 9.93 (d, J=6.19 Hz, 1H), 10.23 (d, J=1.90 Hz, 1H).

### Preparation of 3-(4-pyrimidin-2-ylpyridazin-1-ium-1-yl)propanoic acid chloride (7E)

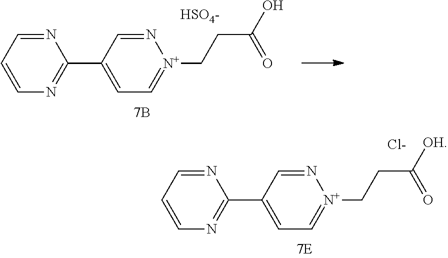 [SMILES: O=C(O)CC[n+]1ccc(-c2ncccn2)cn1 ; "HSO4-" ; O=C(O)CC[n+]1ccc(-c2ncccn2)cn1 ; [Cl-]]

General Procedure 6—Salt Exchange, Amberlite Salt Switch Method

To a three neck round bottom flask (250 mL), charge water (80 mL) at 24° C. under N₂ atmosphere. Start stirring at 300-400 rpm. Charge 3-(4-pyrimidin-2-ylpyridazin-1-ium-1-yl)propanoic acid hydrogen sulfate (7B) (8.32 g, 23.10 mmol, 1 eq.). Charge amberlite resin (IRN78 hydroxide form) (4.67 g, 2.00 eq. g, 47.40 mmol, 2.00 eq.) at 24° C. and stir for 10 min. Filter the resulting suspension on sintered funnel. Wash the resin bed with water (3×10 mL) and combine the aqueous layers. Add conc. HCl (2.53 g, 24.30 mmol, 1.00 eq., 35% in H₂O) to the aqueous layer and stir for 30 min at 24° C. Concentrate the acidic solution at 50° C. under reduced pressure to afford 7E as off white solid (5.77 g, 92% yield from 7B, 98% assay).

7E: ¹H NMR (400 MHz, D₂O) 6 ppm: 3.33 (t, J=6.03 Hz, 2H), 5.20 (t, J=6.03 Hz, 2H), 7.73 (t, J=5.00 Hz, 1H), 9.08 (d, J=5.08 Hz, 2H), 9.27 (dd, J=6.19, 2.22 Hz, 1H), 9.94 (d, J=6.19 Hz, 1H), 10.25 (s, 1H).

General Procedure 7—Salt Exchange, BaCl₂ Salt Switch Method

To a three neck round bottom flask (250 mL), charge aqueous layer containing ethyl 3-(4-pyrimidin-2-ylpyridazin-1-ium-1-yl)propanoate hydrogen sulfate (7A), and 3-(4-pyrimidin-2-ylpyridazin-1-ium-1-yl)propanoic acid hydrogen sulfate (7B) (103.10 g, 68% w/w of 7A and 7.8% w/w of 7B) (273.13 g, 6% strength) at 24° C. under N₂ atmosphere. Start stirring at 300-400 rpm. Dose BaCl₂ (66.80 g, 1.00 eq. 1M solution) over the period of 5-7 min. Heat the reaction mixture at 95-100° C. for 120-180 min and monitor the progress on UFLC. Cool to 95-100° C. and filter the precipitated BaSO₄ over Celite® bed. Distill out the aqueous under 40 mbar at Tᵢ=60° C. to keep 0.75 volume in the flask. Add isopropanol (62 mL) and acetone (16 mL) and stir for 10-15 min. Cool to 10° C. in 30 min and continue stirring for 60 min. Filter off the desired product 7E as off-white solid (11.52 g, 62.00% yield (from 6A), 97% assay).

7E: ¹H NMR (400 MHz, D₂O) δ ppm: 3.33 (t, J=6.03 Hz, 2H), 5.20 (t, J=6.03 Hz, 2H), 7.73 (t, J=5.00 Hz, 1H), 9.08 (d, J=5.08 Hz, 2H), 9.27 (dd, J=6.19, 2.22 Hz, 1H), 9.94 (d, J=6.19 Hz, 1H), 10.25 (s, 1H).

### tert-butyl 3-(6-oxo-4-pyrimidin-2-yl-pyridazin-1-yl)propanoate (5D)

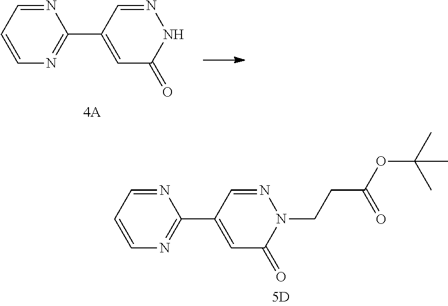 [SMILES: O=c1cc(-c2ncccn2)cn[nH]1 ; CC(C)(C)OC(=O)CCn1ncc(-c2ncccn2)cc1=O]

5D Can be prepared from 4A via general alkylation procedure 1 using tert-butyl prop-2-enoate. 5D: ¹H NMR (400 MHz, CDCl₃) δ ppm 1.46 (s, 9H) 2.82 (t, J=7.15 Hz, 2H) 4.50 (t, J=7.15 Hz, 2H) 7.37 (t, J=4.95 Hz, 1H) 7.93 (d, J=2.20 Hz, 1H) 8.76 (d, J=2.20 Hz, 1H) 8.89 (d, J=4.77 Hz, 2H)

### 3-(6-oxo-4-pyrimidin-2-yl-pyridazin-1-yl)propanoic acid (5B)

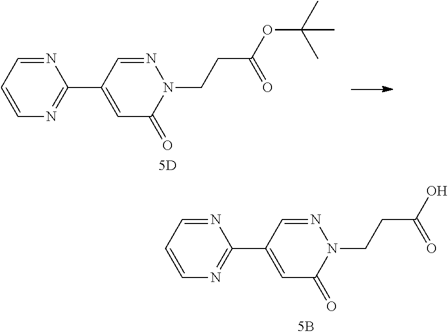 [SMILES: CC(C)(C)OC(=O)CCn1ncc(-c2ncccn2)cc1=O ; O=C(O)CCn1ncc(-c2ncccn2)cc1=O]

5B Can be prepared from 5D via general hydrolysis procedures well known in the art.

¹H NMR (400 MHz, D6-DMSO) δ ppm 2.75 (t, J=7.34 Hz, 2H) 4.32 (t, J=7.15 Hz, 2H) 7.66 (t, J=4.95 Hz, 1H) 7.66 (d, J=2.20 Hz, 1H) 8.69 (d, J=2.20 Hz, 1H) 9.03 (d, J=4.77 Hz, 2H) 11.9 (bs, 1H)

### tert-butyl 3-(4-pyrimidin-2-yl-6-thioxo-pyridazin-1-yl)propanoate 6D

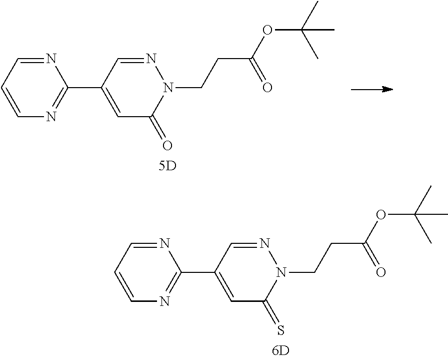 [SMILES: CC(C)(C)OC(=O)CCn1ncc(-c2ncccn2)cc1=O ; CC(C)(C)OC(=O)CCn1ncc(-c2ncccn2)cc1=S]

Prepared from 5D via general procedure 2 in 83% yield.

¹H NMR (400 MHz, CDCl₃) δ ppm 1.48 (s, 9H) 2.95 (t, J=7.15 Hz, 2H) 4.95 (t, J=7.15 Hz, 2H) 7.38 (t, J=4.95 Hz, 1H) 8.76 (d, J=2.57 Hz, 1H) 8.89 (d, J=5.14 Hz, 2H) 9.04 (d, J=2.20 Hz, 1H)

### tert-butyl 3-(4-pyrimidin-2-ylpyridazin-1-ium-1-yl)propanoate hydrogen sulfate 7D

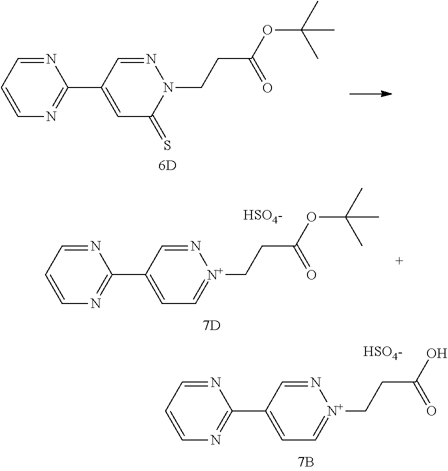

Prepared from 6D via general desulfurization procedure 4 in 57% yield as a solid.

7D: ¹H NMR (400 MHz, D₂O) δ ppm 1.38 (s, 9H) 3.24 (t, J=6.3 Hz, 2H) 5.17 (t, J=6.3 Hz, 2H) 7.72 (t, J=5.03 Hz, 1H) 9.06 (d, J=5 Hz, 2H) 9.29 (dd, J=5.53, 2.2 Hz, 1H) 9.91 (d, J=5.53 Hz, 1H) 10.25 (d, J=1.8 Hz, 1H)

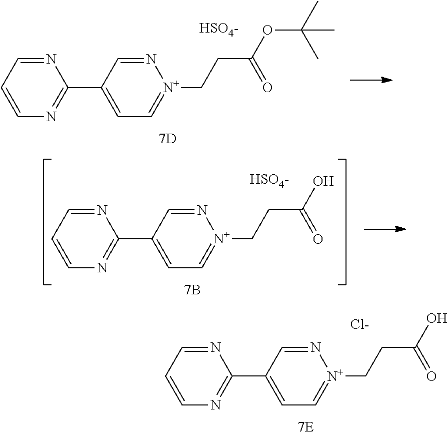 [SMILES: CC(C)(C)OC(=O)CC[n+]1ccc(-c2ncccn2)cn1 ; "HSO4-" ; O=C(O)CC[n+]1ccc(-c2ncccn2)cn1 ; "HSO4-" ; O=C(O)CC[n+]1ccc(-c2ncccn2)cn1 ; [Cl-]]

7D can be converted to 7E by telescoping general procedures 5 and 6 in 92% yield without isolation of 7B.

### Preparation of 3-(4-pyrimidin-2-yl-6-thioxo-pyridazin-1-yl)propanenitrile (6C)

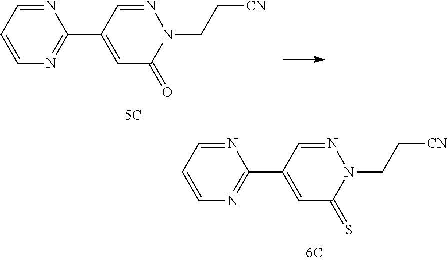 [SMILES: N#CCCn1ncc(-c2ncccn2)cc1=O ; N#CCCn1ncc(-c2ncccn2)cc1=S]

Compound 6C was prepared according to the general sulfurization procedure 2.

6C: ¹H NMR (400 MHz, CDCl₃) δ ppm 3.17 (t, J=6.79 Hz, 2H) 4.99 (t, J=6.79 Hz, 2H) 7.40 (t, J=4.95 Hz, 1H) 8.75 (d, J=1.83 Hz, 1H) 8.90 (d, J=5.14 Hz, 2H) 9.11 (d, J=2.20 Hz, 1H)

### 3-(4-pyrimidin-2-ylpyridazin-1-ium-1-yl)propanenitrile hydrogen sulfate 7C

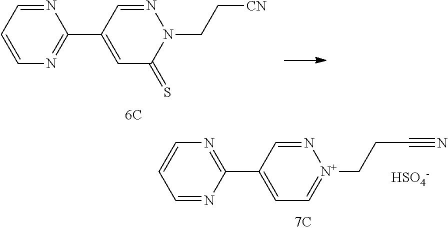

Compound 7C was prepared in 42% yield from 6C via general desulfurization procedure 4.

7C: ¹H NMR (400 MHz, D₂O) δ ppm 3.40 (t, J=6.24 Hz, 2H) 5.22 (t, J=6.24 Hz, 2H) 7.67 (t, J=4.95 Hz, 1H) 9.02 (d, J=4.77 Hz, 2H) 9.17 (dd, J=5.87, 1.83 Hz, 1H) 9.91 (d, J=6.24 Hz, 1H) 10.26 (bs, 1H)

### 3-(4-pyrimidin-2-yl-6-thioxo-pyridazin-1-yl)propanoic acid 6B

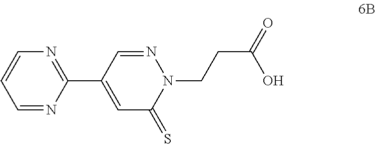 [SMILES: O=C(O)CCn1ncc(-c2ncccn2)cc1=S]

General Procedure 9—HCl Hydrolysis

To a solution or tert-butyl 3-(4-pyrimidin-2-yl-6-thioxo-pyridazin-1-yl)propanoate (0.105 g, 0.33 mmol) in dioxane (1.65 mL) was added HCl 2M (6.59 mL, 7.85 g, 13.2 mmol). The reaction mixture was heated to reflux for 15 mins and then concentrated in vacuo to afford 3-(4-pyrimidin-2-yl-6-thioxo-pyridazin yl)propanoic acid 6B (0.083 g, 0.32 mmol, 96% yield).

1H NMR (400 MHz, CD₃OD) δ=9.08 (d, J=2.2 Hz, 1H), 8.94 (d, J=4.9 Hz, 2H), 8.61 (d, J=2.2 Hz, 1H), 7.52 (t, J=4.9 Hz, 1H), 4.89 (t, J=7.2 Hz, 2H), 2.99 (t, J=7.2 Hz, 2H)

### Preparation of Ethyl 3-(6-oxo-4-pyridazin-3-yl-pyridazin-1-yl)propanoate (15A)

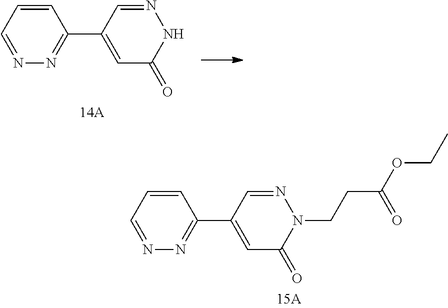 [SMILES: O=c1cc(-c2cccnn2)cn[nH]1 ; CCOC(=O)CCn1ncc(-c2cccnn2)cc1=O]

To a three neck round bottom flask (250 mL), charge 2-methyltetrahydrofuran (100 mL) at 24° C. under N₂ atmosphere. Start stirring at 400 rpm. Charge 4-pyridazin-3-yl-1H-pyridazin-6-one 14A (10.00 g, 52.91 mmol). Charge K₂CO₃ (0.40 eq., 10.58 mmol) followed by tetrabutylammonium bromide (0.05 eq., 2.65 mmol). Heat the reaction mixture to 80° C. Charge ethyl prop-2-enoate (12.82 g, 2.40 eq., 127.0 mmol) dropwise with syringe pump over a period of 1 h. Continue the reaction at 80° C. for 420 min with monitoring the progress on UFLC. Cool the reaction to 24° C., add water (100 mL). Extract the aqueous with ethylacetate (2×100 mL) and separate the layers. Evaporate the combined ethylacetate layers to give pale violet solid (15.00 g). Triturate the pale violet solid in TBME (45 mL) to give the desired compound ethyl 3-(6-oxo-4-pyridazin-3-yl-pyridazin-1-yl)propanoate (15A) (13.50 g, 87% yield, 94% assay).

15A: ¹H NMR (400 MHz, DMSO-d₆) δ ppm 1.16 (t, J=7.06 Hz, 3H), 2.82 (t, J=6.98 Hz, 2H), 4.06 (q, J=7.14 Hz, 2H), 4.35 (t, J=6.90 Hz, 2H), 7.67 (d, J=2.06 Hz, 1H), 7.89 (dd, J=8.64, 5.00 Hz, 1H), 8.41 (dd, J=8.64, 1.51 Hz, 1H), 8.70 (d, J=2.22 Hz, 1H), 9.34 (dd, J=4.92, 1.43 Hz, 1H).

### Preparation of Ethyl 3-(4-pyridazin-3-yl-6-thioxo-pyridazin-1-yl) propanoate (16A)

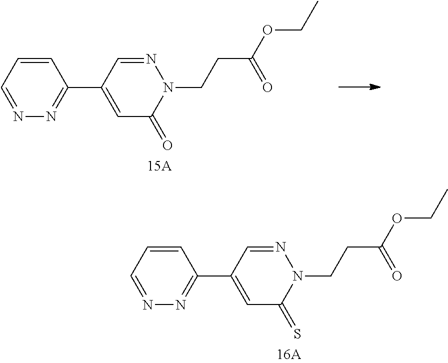 [SMILES: CCOC(=O)CCn1ncc(-c2cccnn2)cc1=O ; CCOC(=O)CCn1ncc(-c2cccnn2)cc1=S]

To a four neck round bottom flask (100 mL), charge pyridine (10.00 mL, 120.00 mmol) at 24° C. under N₂ atmosphere. Start stirring at 250 rpm. Charge P₂S₅ (1.96 g, 0.50 eq., 8.75 mmol) at 24° C. Heat the reaction mixture to 115° C. Charge solution of ethyl 3-(6-oxo-4-pyridazin-3-yl-pyridazin-1-yl)propanoate 15A (5.00 g, 17.5 mmol) in Pyridine (15.10 mL, 190.00 mmol) dropwise over a period of 1 h at 115° C. Distilled out pyridine (15.00 mL, 190.00 mmol) and continue stirring at 115° C. for 240 min. Monitor the progress on HPLC, recharge distilled pyridine (15.00 mL, 190.0 mmol) and cooled the reaction mixture to 60° C., reaction mass quenched by water (37.50 mL) at 24° C., resultant suspension cooled to 20-25° C. Continue stirring at 20-25° C., 60 min. Filtration to give ethyl 3-(4-pyridazin-3-yl-6-thioxo-pyridazin-1-yl) propanoate (16A) as orange solid (4.70 g, 89.00% yield, 97% assay).

16A: ¹H NMR (400 MHz, DMSO-d₆) δ ppm 1.16-1.19 (t, J=4.0 Hz, 3H), 2.96-2.99 (t, J=4.0 Hz, 2H), 4.06-4.11 (q, J=4.0 Hz, 2H), 4.78-4.82 (t, J=4.0 Hz, 2H), 7.89-7.92 (dd, J=8.0, 4.0 Hz, 1H), 8.46-8.52 (m, 2H), 9.11 (m, 1H), 9.35-9.37 (m, 1H)

### Preparation of Ethyl 3-(4-pyridazin-3-ylpyridazin-1-ium-1-yl)propanoate hydrogen sulfate (17A)

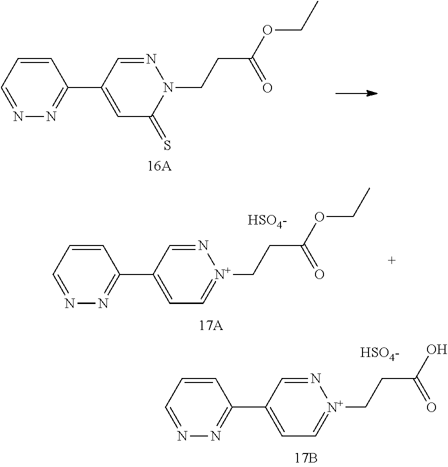

To a four neck round bottom flask (100 mL), charge ethyl acetate/Water/Formic acid (4:2:1) (35.00 mL) at 24° C. under N₂ atmosphere. Start stirring at 275 rpm. Charge ethyl 3-(4-pyridazin-3-yl-6-thioxo-pyridazin-1-yl)propanoate (16A) (5.00 g, 16.00 mmol) at 24° C. Charge H₂O₂ (5.70 g, 5.10 mL, 3.06 eq., 50.00 mmol, 30% in H₂O) dropwise in 240 min at 20-25° C. under N₂ atmosphere. Continue the reaction at 20-25° C. for another 120 min and monitor the progress on UFLC. Charge Water (40 mL) and separate the organic layer. Extract the aqueous layer with ethyl acetate (4×15 mL) and separate the layers. Treat the aqueous layer with activated charcoal (500.00 mg, 10% w/w) and continue stirring overnight at 20-25° C. Filter the aqueous solution through Celite® bed to give a clear solution of ethyl 3-(4-pyridazin-3-ylpyridazin-1-ium-1-yl)propanoate hydrogen sulfate (17A) (49.00 g, 12.70 mmol, 78% yield, 9.2% w/w in H₂O) and 3-(4-pyridazin-3-ylpyridazin-1-ium-1-yl)propanoic acid hydrogen sulfate (17B) (49.0 g, 3.6 mmol, 3.6%, 2.4 w/w in H₂O %). The aqueous solution was used as such for next step.

17A: ¹H NMR (400 MHz, DMSO-d₆) δ ppm 10.12-10.13 (m, 1H), 9.92-9.90 (m, 1H), 9.36-9.34 (m, 1H), 9.15-9.11 (m, 1H), 8.57-8.54 (m, 1H), 8.06-8.02 (m, 1H), 5.18-5.13 (m, 2H), 4.07 (q, J=8.0 Hz, 2H), 3.27 (t, J=8.0 Hz, 2H), 1.12 (t, J=8.0 Hz, 3H).

17A was Also Prepared According to the Following Procedure:

To a four neck round bottom flask (100 mL), charge acetic acid (21 mL, 1.5 M) at 24° C. under N₂ atmosphere. Start stirring at 300-400 rpm. Charge ethyl 3-(4-pyridazin-3-yl-6-thioxo-pyridazin-1-yl)propanoate (16 A) (1.00 g, 3.20 mmol, 1.00 eq., 93.00 mass %) at 24° C. and stir for 10 min. Dose H₂O₂ (1.20 mL, 11.00 mmol, 3.30 eq., 27.00 mass %) over the period of 120 min. Stir the reaction for 60 min and monitor the progress on HPLC. Quench the unreacted H₂O₂ into the reaction with saturated Na₂SO₃ (0.12 g, 0.96 mmol, 0.30 eq., 98.00 mass % in water) solution and stir for 30 min at 24° C. Concentrate the reaction mass under rota vapour to get crude gummy liquid (1.87 g, 67.00% ethyl 3-(4-pyridazin-3-ylpyridazin-1-ium-1-yl)propanoate hydrogen sulfate (17 A), 41.00 mass %).

### Preparation of 3-(4-pyridazin-3-ylpyridazin-1-ium-1-yl)propanoic acid hydrogen sulfate (17B)

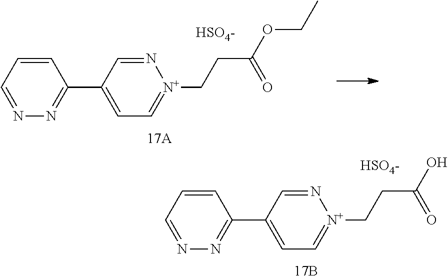 [SMILES: CCOC(=O)CC[n+]1ccc(-c2cccnn2)cn1 ; "HSO4-" ; O=C(O)CC[n+]1ccc(-c2cccnn2)cn1 ; "HSO4-"]

To a four neck round bottom flask (100 mL) installed with Dean-Stark and water condenser, charge ethyl 3-(4-pyridazin-3-ylpyridazin-1-ium-1-yl)propanoate;hydrogen sulfate 17A (49.00 g, 12.70 mmol, 9.2% w/w in H₂O) at 24° C. Start stirring at 275 rpm. Charge conc. hydrogen chloride (0.662 g, 0.50 eq., 6.35 mmol, 35% in H₂O) at 24° C. Heat the reaction mixture to 100° C. Continue the reaction at 100° C. for 3 h and monitor the progress on UFLC. Cool the reaction mixture to 24° C. Evaporate (˜10 mL) the aqueous layer to give ethyl 3-(4-pyridazin-3-ylpyridazin-1-ium-1-yl)propanoate hydrogen sulfate (17B) (47.00 g, 11.10 mmol, 67% yield from 16A, 7.66% w/w in H₂O). The aqueous solution was used as such for next step.

17B: ¹H NMR (400 MHz, DMSO-d₆) δ ppm 3.30 (t, J=4 Hz, 2H), 5.17 (t, J=4.0 Hz, 2H), 8.09 (dd, J=8.0, 4.0 Hz, 1H), 8.59 (dd, J=8.0, 4.0 Hz, 1H), 9.14-9.16 (m, 1H), 9.39-9.40 (m, 1H), 9.93 (d, J=8.0 Hz, 1H), 10.16 (d, J=8.0 Hz, 1H).

### 3-(4-Pyridazin-3-ylpyridazin-1-ium-1-yl)propanoic acid chloride (17E)

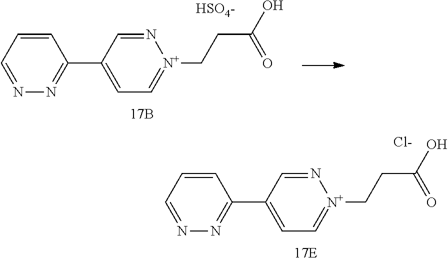 [SMILES: O=C(O)CC[n+]1ccc(-c2cccnn2)cn1 ; "HSO4-" ; [Cl-] ; O=C(O)CC[n+]1ccc(-c2cccnn2)cn1]

To a four neck round bottom flask (100 mL), charge ethyl 3-(4-pyridazin-3-ylpyridazin-1-ium-1-yl)propanoate hydrogen sulfate (17B) (47.00 g, 11.10 mmol, 7.66% w/w in H₂O). Start stirring at 275 rpm. Charge Amberlite IRN78 hydroxide form (38.00 g, 69.40 mmol) over a period of 60 min until pH becomes 7-8 at 24° C. Filter the resulting suspension on sintered funnel. Wash the resin bed with water (2×15 mL) and combine the aqueous layers. Add conc. HCl (1.14 g, 1.00 eq., 11.00 mmol, 35% in H₂O) to the aqueous layer and stir for 30 min at 24° C. Concentrate the acidic solution at 50° C. under reduced pressure to afford crude 17E (3.10 g, 62% yield from 16A, 86% assay). Crystallization in water/iPrOH/acetone (1:4:3, 21.2 mL) to afford 17E (1.76 g, 39% yield from 16A, 98% assay).

17E: ¹H NMR (400 MHz, D₂O) δ ppm 10.15 (m, 1H), 9.93 (d, J=8.0 Hz, 1H), 9.37-9.35 (m, 1H), 9.15-9.13 (m, 1H), 8.57-8.54 (m, 1H), 8.06-8.02 (m, 1H), 5.17 (t, J=8.0 Hz, 2H), 3.30 (t, J=8.0 Hz, 2H).

17E was Also Prepared from 17B Via the Following Procedure:

To a three neck round bottom flask (250 mL) assemble with dean-stark and water condenser. Start stirring at 400 rpm. A mixture of ethyl 3-(4-pyridazin-3-ylpyridazin-1-ium-1-yl)propanoate hydrogen sulfate (17A) (1.00 eq., 139.00 g, 36.50 mmol, 9.36% w/w in H₂O) and 3-(4-pyridazin-3-ylpyridazin ium-1-yl)propanoic acid hydrogen sulfate 17B (139.00 g, 2.60 mmol, 0.61% w/w in H₂O) was charged. The mixture was stirred for 5 min. Charge aqueous solution of BaCl₂.2H₂O (41.00 mL, 1.05 eq., 41.00 mmol, 1M solution in water) dropwise over a period of 10 min at room temperature. Continue the reaction at room temperature for 20 min and monitor the progress using BaCl₂ test. Heat the reaction mixture to 100° C. Continue the reaction at 100° C. for 180 min and monitor the progress on UFLC. Cool the reaction to 24° C. Filter the aqueous solution through the Celite® bed and wash the Celite® bed with water (3×45 mL). Evaporate aqueous solution to give 17E crude as brown solid (12.70 g). Crystallization in water/iPrOH (1:4, 100 mL) to afford 17E as an off-white solid (7.80 g, 69% yield from 16A, 94% assay).

17E: ¹H NMR (400 MHz, D₂O) δ ppm 10.15 (m, 1H), 9.93 (d, J=8.0 Hz, 1H), 9.37-9.35 (m, 1H), 9.15-9.13 (m, 1H), 8.57-8.54 (m, 1H), 8.06-8.02 (m, 1H), 5.17 (t, J=4.0 Hz, 2H), 3.30 (t, J=4.0 Hz, 2H).

### Preparation of tert-butyl 3-(6-oxo-4-pyridazin-3-yl-pyridazin-1-yl)propanoate (15D)

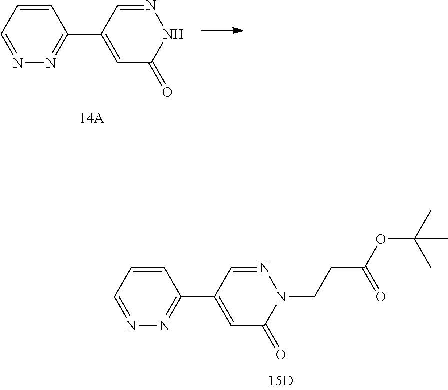 [SMILES: O=c1cc(-c2cccnn2)cn[nH]1 ; CC(C)(C)OC(=O)CCn1ncc(-c2cccnn2)cc1=O]

To a three neck round bottom flask (25 mL), charge acetonitrile (8 mL) at 24° C. under N₂ atmosphere. Start stirring at 400 rpm. Charge 4-pyridazin-3-yl-1H-pyridazin-6-one 14A (1.00 g, 5.45 mmol) and stir for 5 min. Charge K₂CO₃ (1.20 eq. 6.55 mmol), followed by tetrabutylammonium bromide (0.05 eq., 0.27 mmol). Heat the reaction mixture to 80° C. Charge tert-butyl prop-2-enoate (0.85 g, 1.20 eq., 6.55 mmol) dropwise in 5 min. Continue the reaction at 80° C. for 240 min with monitoring the progress on UFLC. Cool the reaction to 24° C., concentrate the acetonitrile and add water (10 mL). Extract the aqueous with ethylacetate (2×10 mL) and separate the layers. Evaporate the combined ethylacetate layers to give pale violet solid (1.38 g). Triturate the pale violet solid in TBME (5 mL) to give the desired compound tert-butyl 3-(6-oxo-4-pyridazin-3-yl-pyridazin-1-yl)propanoate (15D) (1.27 g, 74% yield, 96% assay).

15D: ¹H NMR (400 MHz, DMSO-d₆) δ ppm 1.36 (s, 9H), 2.73 (t, J=6.85 Hz, 2H), 4.31 (t, J=6.91 Hz, 2H), 7.67 (d, J=2.20 Hz, 1H), 7.90 (dd, J=8.68, 5.01 Hz, 1H), 8.41 (dd, J=8.62, 1.41 Hz, 1H), 8.70 (d, J=2.20 Hz, 1H), 9.34 (dd, J=4.95, 1.41 Hz, 1H).

### tert-butyl 3-(4-pyridazin-3-yl-6-thioxo-pyridazin-1-yl)propanoate 16D

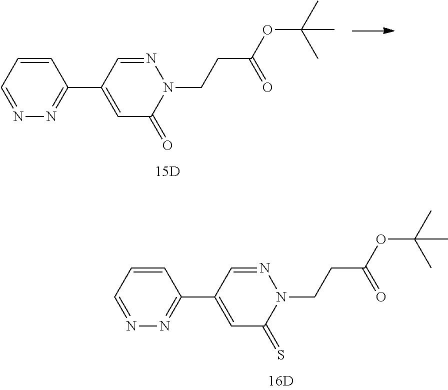 [SMILES: CC(C)(C)OC(=O)CCn1ncc(-c2cccnn2)cc1=O ; CC(C)(C)OC(=O)CCn1ncc(-c2cccnn2)cc1=S]

16D was prepared from 15D in 32% yield according to General Procedure 2.

16D: ¹H NMR (400 MHz, DMSO-d₆) δ ppm 1H NMR (400 MHz, DMSO-d₆) δ ppm 1.39 (s, 9H), 2.89 (t, J=6.74 Hz, 2H), 4.76 (t, J=6.82 Hz, 2H), 7.90 (dd, J=8.64, 5.00 Hz, 1H), 8.47 (d, J=2.22 Hz, 1H), 8.52 (dd, J=8.64, 1.51 Hz, 1H), 9.11 (d, J=2.22 Hz, 1H), 9.36 (dd, J=4.92, 1.43 Hz, 1H)

### tert-butyl 3-(4-pyridazin-3-ylpyridazin-1-ium-1-yl)propanoate hydrogen sulfate 17D

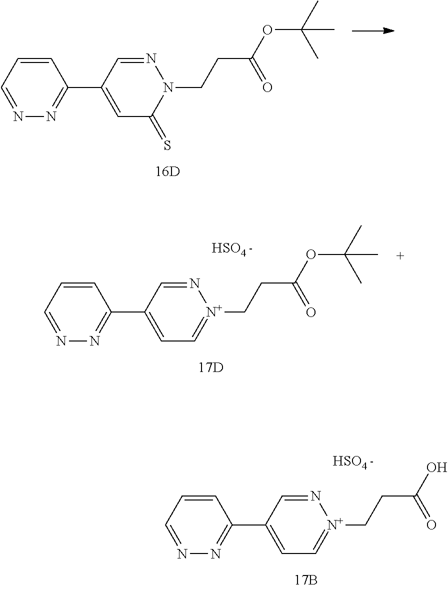 [SMILES: CC(C)(C)OC(=O)CCn1ncc(-c2cccnn2)cc1=S ; CC(C)(C)OC(=O)CC[n+]1ccc(-c2cccnn2)cn1 ; "HSO4" ; O=C(O)CC[n+]1ccc(-c2cccnn2)cn1 ; "HSO4"]

17D was prepared from 16D in 56% yield according to general desulfurization procedure 4:

17D: ¹H NMR (400 MHz, D₂O) δ ppm 1.35 (s, 9H), 3.21 (t, J=6.11 Hz, 2H), 5.15 (t, J=6.03 Hz, 2H), 8.04 (dd, J=8.64, 5.15 Hz, 1H), 8.55 (dd, J=8.72, 1.43 Hz, 1H), 9.16 (dd, J=6.34, 2.54 Hz, 1H), 9.36 (dd, J=5.15, 1.51 Hz, 1H), 9.91 (d, J=6.34 Hz, 1H), 10.17 (d, J=2.06 Hz, 1H)

### 3-(4-pyridazin-3-ylpyridazin-1-ium-1-yl)propanoic acid hydrogen sulfate 17B

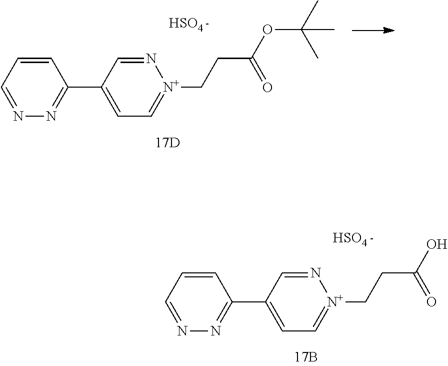 [SMILES: CC(C)(C)OC(=O)CC[n+]1ccc(-c2cccnn2)cn1 ; "HSO4" ; O=C(O)CC[n+]1ccc(-c2cccnn2)cn1 ; "HSO4"]

To a four neck round bottom flask (5.0 L), charge solution of tert-butyl 3-(4-pyridazin-3-ylpyridazin-1-ium-1-yl)propanoate;methane (17D) (870 g, 1358 mmol, 60.00 mass %) in water. Start stirring at 275 rpm. Dose conc. HCl (180.0 g, 1630 mmol, 33 mass %) at temperature 22-25° C. Heat the reaction mass at 50° C. and stir for 3 h. Monitor the progress on HPLC. After completion of reaction, the reaction mixture was concentrated to give crude 3-(4-pyridazin-3-ylpyridazin-1-ium-1-yl)propanoic acid hydrogen sulfate (17 B) (517 g, 1181 mmol, 86%, 75.00 mass %). The aq. solution was used as such without further purification.

17B: ¹H NMR (400 MHz, DMSO-d₆) δ ppm 3.30 (t, J=4 Hz, 2H), 5.17 (t, J=4.0 Hz, 2H), 8.09 (dd, J=8.0, 4.0 Hz, 1H), 8.59 (dd, J=8.0, 4.0 Hz, 1H), 9.14-9.16 (m, 1H), 9.39-9.40 (m, 1H), 9.93 (d, J=8.0 Hz, 1H), 10.16 (d, J=8.0 Hz, 1H).

### 3-(4-Pyridazin-3-ylpyridazin-1-ium-1-yl)propanoic acid chloride (17E)

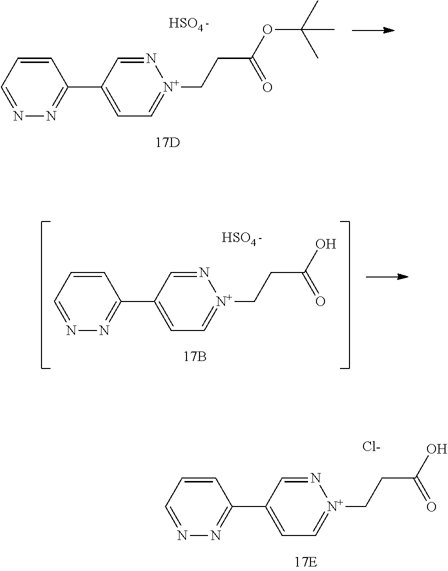 [SMILES: CC(C)(C)OC(=O)CC[n+]1ccc(-c2cccnn2)cn1 ; "HSO4" ; O=C(O)CC[n+]1ccc(-c2cccnn2)cn1 ; "HSO4" ; [Cl-] ; O=C(O)CC[n+]1ccc(-c2cccnn2)cn1]

17D can be converted to 17E by telescoping general procedures 5 and 6 in 90% yield (90% assay) without isolation of 17B.

### Preparation of 4-pyridazin-3-yl-1H-pyridazine-6-thione (15E)

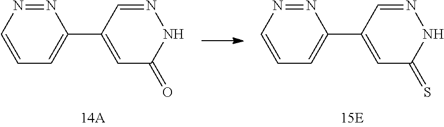 [SMILES: O=c1cc(-c2cccnn2)cn[nH]1 ; S=c1cc(-c2cccnn2)cn[nH]1]

4-pyridazin-3-yl-1H-pyridazin-6-one (14A) (2.00 g) was mixed with dry pyridine (16 ml) and the reaction mixture was heated to 90° C. under stirring. Phosphorus pentasulfide (1.27 g) was added in portions and the reaction mixture was stirred at 90° C. for 6 h and for additional 2 h at 110° C. The reaction was cooled to 5° C. and ice cold water (100 ml) was added under cooling. The suspension was heated to 60° C. and slowly cooled to RT. The solid was filtered, washed twice with ice cold water, and dried under reduced pressure providing 4-pyridazin-3-yl-1H-pyridazine-6-thione 15E (2.08 g) as a yellow solid. 15E: ¹H NMR (DMSO-d₆) δ=14.98 (br s, 1H), 9.37 (dd, J=4.77, 1.47 Hz, 1H), 9.07 (d, J=2.20 Hz, 1H), 8.50 (dd, J=8.44, 1.47 Hz, 1H), 8.32 (d, J=2.20 Hz, 1H), 7.91 (dd, J=8.62, 4.95 Hz, 1H) LC-MS RT (Standard Method): 0.28 min; MS (ES-pos) calcd for [C8H6N4S]+H⁺: 191, found 191.

### Preparation of 3-(4-pyridazin-3-yl-6-thioxo-pyridazin-1-yl)propanenitrile (16C)

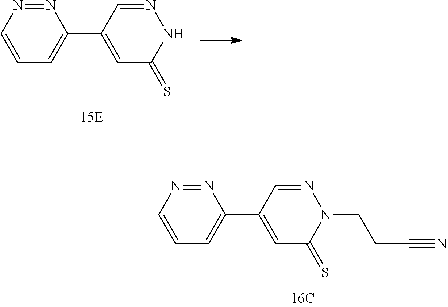 [SMILES: S=c1cc(-c2cccnn2)cn[nH]1 ; N#CCCn1ncc(-c2cccnn2)cc1=S]

A flask was charged with 4-pyridazin-3-yl-1H-pyridazine-6-thione (15E) (6.20 g, 30 mmol, 1.00 eq) and dissolved in Me-THF (89 mL). Tetrabutylammonium hydroxide (1.20 g, 1M in MeOH, 1.5 mmol, 0.05 equiv.) and Acrylonitrile (1.7 g, 31 mmol, 1.00 eq) were added. The resulting mixture was stirred at 50° C. After 2 h, tetrabutylammonium hydroxide (0.25 g, 1M in MeOH, 1.5 mmol, 0.01 equiv.) and Acrylonitrile (1.7 g, 31 mmol, 1.00 eq) were again added and the mixture was stirred at 50° C. overnight.

The mixture was then filtered and the resulting solid was washed with 200 mL of TBME (200 mL).

The solid was dried in vacuum for 30 min to give 3-(4-pyridazin-3-yl-6-thioxo-pyridazin-1-yl)propanenitrile 16C isolated as a brown solid (6.74 g, 86% purity, 80% yield).

16C: ¹H NMR (DMSO-d₆) δ=9.38 (br d, J=4.03 Hz, 1H), 9.19 (br d, J=1.47 Hz, 1H), 8.54 (br d, J=8.44 Hz, 1H), 8.50 (br d, J=1.47 Hz, 1H), 7.92 (br dd, J=8.44, 5.14 Hz, 1H), 4.87 (br t, J=6.24 Hz, 2H), 3.25 (br t, J=6.24 Hz, 2H)

LC-MS RT (Standard Method): 0.59 min; MS (ES-pos) calcd for [C11H9N5S]+H⁺: 244, found 244.

### Preparation of 3-(4-pyridazin-3-yl-6-thioxo-pyridazin-1-yl)propanoic acid (16B)

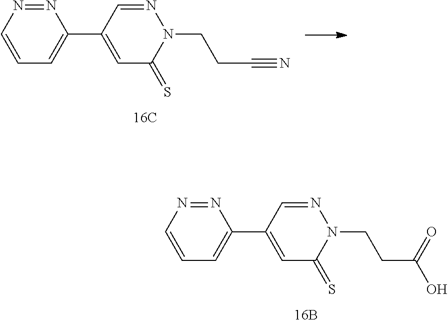 [SMILES: N#CCCn1ncc(-c2cccnn2)cc1=S ; O=C(O)CCn1ncc(-c2cccnn2)cc1=S]

3-(4-pyridazin-3-yl-6-thioxo-pyridazin-1-yl)propanenitrile (16C) (0.5 g) was dissolved in hydrochloric acid (4 M, 4.8 ml). The reaction was stirred at 50° C. for 6 h, diluted with water and filtered. The solid was washed with water, and dried under reduced pressure providing 3-(4-pyridazin-3-yl-6-thioxo-pyridazin-1-yl)propanoic acid 16B (0.39 g, 96% assay, 75% yield) as a brown solid.

16B: ¹H NMR (DMSO-d₆) δ=12.49 (br s, 1H), 9.37 (dd, J=4.77, 1.47 Hz, 1H), 9.13 (d, J=2.20 Hz, 1H), 8.51 (dd, J=8.80, 1.47 Hz, 1H), 8.47 (d, J=2.57 Hz, 1H), 7.91 (dd, J=8.80, 5.14 Hz, 1H), 4.78 (t, J=7.15 Hz, 2H), 2.92 (t, J=6.97 Hz, 2H)

LC-MS RT (STANDARD LONG Method): 1.49 min; MS (ES-pos) calcd for [C11H10N4O2S]+H⁺: 263, found 263.

### Preparation of 3-(4-pyridazin-3-yl-6-thioxo-pyridazin-1-yl)propanamide (16E)

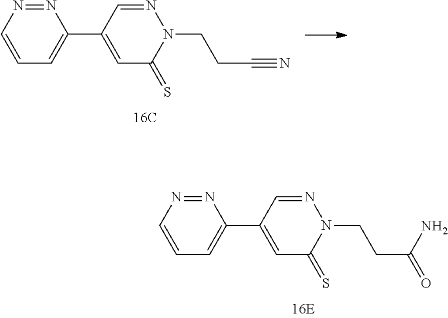 [SMILES: N#CCCn1ncc(-c2cccnn2)cc1=S ; NC(=O)CCn1ncc(-c2cccnn2)cc1=S]

3-(4-pyridazin-3-yl-6-thioxo-pyridazin-1-yl)propanenitrile (16C) (0.54 g) was dissolved in acetic acid (6.2 ml) and hydrochloric acid (4 M, 1.8 ml) was added. The reaction was stirred at RT for 3 h, diluted with water (18 ml) and filtered. The solid was washed with water, and dried under reduced pressure providing 3-(4-pyridazin-3-yl-6-thioxo-pyridazin-1-yl)propanamide 16E (0.32 g) as a brown solid.

16E: ¹H NMR (DMSO-d₆) δ=9.36 (br d, J=5.14 Hz, 1H), 9.10 (d, J=1.83 Hz, 1H), 8.50 (br d, J=8.44 Hz, 1H), 8.44 (d, J=1.83 Hz, 1H), 7.90 (dd, J=8.62, 4.95 Hz, 1H), 7.50 (br s, 1H), 6.97 (br s, 1H), 4.78 (br t, J=7.34 Hz, 2H), 2.75 (br t, J=7.52 Hz, 2H)

LC-MS RT (STANDARD LONG Method): 1.32 min; MS (ES-pos) calcd for [C11H11N5OS]+H⁺: 262, found 262.

### Preparation of 3-(4-pyridazin-3-ylpyridazin-1-ium-1-yl)propanamide hydrogensulfate (17E)

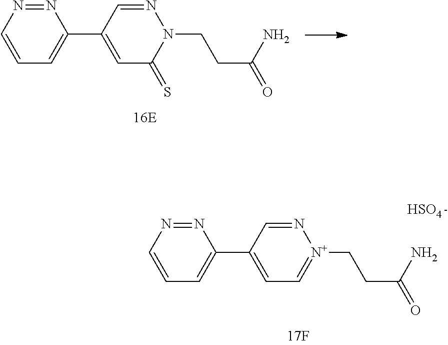 [SMILES: NC(=O)CCn1ncc(-c2cccnn2)cc1=S ; "HSO4" ; NC(=O)CC[n+]1ccc(-c2cccnn2)cn1]

3-(4-pyridazin-3-yl-6-thioxo-pyridazin-1-yl)propanamide (16E) (30 mg) was dissolved in acetic acid (0.57 ml) and hydrochloric acid (4 M, 0.057 ml). Hydrogen peroxide (35% in water, 0.33 ml) was added. The reaction was stirred at RT for 0.5 h, diluted with 2-Propanol (4 ml) and filtered. The solid was washed with 2-Propanol, and dried under reduced pressure providing 3-(4-pyridazin-3-ylpyridazin-1-ium-1-yl)propanamide hydrogensulfate 17F (18 mg).

17F: ¹H NMR (D₂O) δ=10.12 (d, J=1.83 Hz, 1H), 9.86 (d, J=6.24 Hz, 1H), 9.35 (dd, J=5.14, 1.47 Hz, 1H), 9.11 (dd, J=6.24, 2.57 Hz, 1H), 8.55 (dd, J=8.44, 1.47 Hz, 1H), 8.04 (dd, J=8.80, 5.14 Hz, 1H), 5.13 (t, J=6.24 Hz, 2H), 3.17 (t, J=6.24 Hz, 2H)

LC-MS RT (STANDARD LONG Method): 0.63 min; MS (ES-pos) calcd for [C11H12N5O]⁺: 230, found 230.

### Preparation of 3-(4-pyridazin-3-ylpyridazin-1-ium-1-yl)propanoic acid hydrogensulfate (17B)

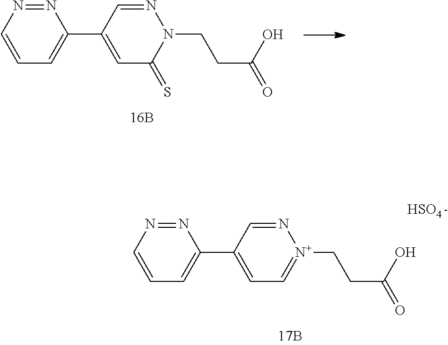 [SMILES: O=C(O)CCn1ncc(-c2cccnn2)cc1=S ; "HSO4" ; O=C(O)CC[n+]1ccc(-c2cccnn2)cn1]

3-(4-pyridazin-3-yl-6-thioxo-pyridazin-1-yl)propanoic acid (16B) (0.30 g) was suspended in acetic acid (5 ml), cooled to 10° C., and hydrogen peroxide (35% in water, 0.32 ml) was added dropwise. After 50 min, the reaction was warmed to RT and Sodium metabisulfite was added until remaining peroxide was quenched. Isopropanol (5 ml) was added, the precipitate was filtered and dried to give 3-(4-pyridazin-3-ylpyridazin-1-ium-1-yl)propanoic acid hydrogensulfate 17B (280 mg) as a beige solid.

### Preparation of 3-(4-pyridazin-3-ylpyridazin-1-ium-1-yl)propanoic acid hydrogensulfate (17B)

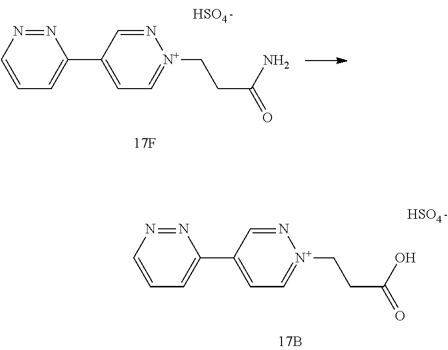 [SMILES: "HSO4" ; NC(=O)CC[n+]1ccc(-c2cccnn2)cn1 ; "HSO4" ; O=C(O)CC[n+]1ccc(-c2cccnn2)cn1]

3-(4-pyridazin-3-ylpyridazin-1-ium-1-yl)propanamide hydrogensulfate 17F (20 mg) was dissolved in hydrochloric acid (4M, 0.5 ml) and stirred at 50° C. for 17 h. The reaction was concentrated and the oily residue was triturated with 2-propanol (4 ml), filtered, and washed with 2-propanol (2×1 ml) to provide 3-(4-pyridazin-3-ylpyridazin-1-ium-1-yl)propanoic acid hydrogensulfate 17B (8 mg) as solid.

### Preparation of 3-(4-pyridazin-3-ylpyridazin-1-ium-1-yl)propanoic acid chloride salt (17E) from 3-(4-pyridazin-3-ylpyridazin-1-ium-1-yl)propanenitrile chloride salt (17G)

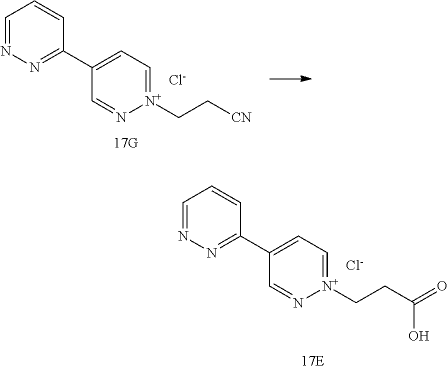 [SMILES: N#CCC[n+]1ccc(-c2cccnn2)cn1 ; [Cl-] ; O=C(O)CC[n+]1ccc(-c2cccnn2)cn1 ; [Cl-]]

3-(4-pyridazin-3-ylpyridazin-1-ium-1-yl)propanenitrile chloride salt 17G (17.9 g, 40.4 mmol, 55.8%) was stirred with hydrochloric acid (46.0 g, 0.404 mol, 10 eq, 32% w/w in H₂O) at 80° C. for 2.5 h. Water (31 g) was added and volatiles (HCl/Water azeotrope) were removed by rotary evaporation at 55° C. To remove excessive HCl as well as water, propionic acid (15.5 g) was added to the residue and the resulting mixture was evaporated to dryness to result in crude product (17G) as a black amorphous (glass-like) solid in 96% yield (24.9 g, purity=41.4%, quantitative 1H NMR in D₂O with 1-Methyl-2-pyridone as standard).

NMR data: ¹H NMR (400 MHz, D₂O) 6 ppm: 10.13 (d, J=2.4 Hz, 1H), 9.95 (d, J=6.3 Hz, 1H), 9.34 (dd, J=5.1 Hz, 1.5 Hz, 1H), 9.15 (dd, J=6.3 Hz, 2.6 Hz, 1H), 8.57 (dd, J=8.7 Hz, 1.5 Hz, 1H), 8.04 (dd, J=8.7 Hz, 5.1 Hz, 1H), 5.18 (t, J=6.1 Hz, 2H), 3.29 (t, J=6.1 Hz, 2H).

### Preparation of 4-pyrimidin-2-yl-1H-pyridazine-6-thione (4C)

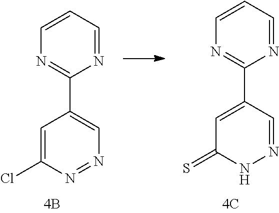 [SMILES: Clc1cc(-c2ncccn2)cnn1 ; S=c1cc(-c2ncccn2)cn[nH]1]

3-chloro-5-pyrimidin-2-yl-pyridazine (4B) (1.00 eq., 0.200 g, 0.997 mmol) and Thiourea (2.00 eq., 0.153 g, 1.99 mmol) were suspended in MeOH (6 mL). The pale-yellow suspension was heated to 60° C. for 2 h. The reaction was after cooled down to room temperature to give a very thick yellow suspension. The solid was filtered off and was washed with a small amount of MeOH. The title compound was obtained after drying in vacuo as a yellow solid (0.167 g, 85% Yield, 97% purity).

1H NMR (DMSO-d6, 400 MHz) 14.99 (br s, 1H), 9.04 (d, J=4.8 Hz, 2H), 9.00 (d, J=1.8 Hz, 1H), 8.30 (s, 1H), 7.67 (t, J=4.9 Hz, 1H)

### Preparation of ethyl 3-(4-pyrimidin-2-ylpyridazin-1-ium-1-yl)propanoate hydrogen sulfate (7A)

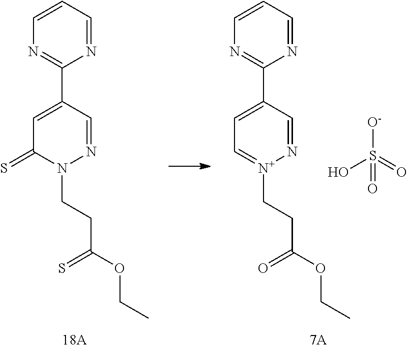 [SMILES: CCOC(=S)CCn1ncc(-c2ncccn2)cc1=S ; CCOC(=O)CC[n+]1ccc(-c2ncccn2)cn1 ; O=S(=O)([O-])O]

O-ethyl 3-(4-pyrimidin-2-yl-6-thioxo-pyridazin-1-yl)propanethioate (18A) (0.500 g, 1.60 mmol) was slurried in Acetic acid (10.7 mL) at 22° C. Additional Acetic acid (3.3 mL) was added to give a clear solution. The resulting brown solution was cooled to 16-18° C. using an ice-water bath. H₂O₂ (30% wt in H₂O) was added in portion via syringe at 16-18° C. as described below.

A first portion of H₂O₂ (30% wt in H₂O, 1.10 eq., 0.2 mL) was added over the period of 1 min at 17-19° C. and stirred for 10 min. A second addition of H₂O₂ (30% wt in H₂O, 0.55 eq., 0.1 mL) was added over the period of 1 min at 17-19° C. and stirred for 10 min. A third addition of H₂O₂ (30% wt in H₂O, 1.65 eq., 0.4 mL) was added over the period of 1 min at 18° C. The cooling ice-water bath was removed and the reaction mixture allowed to gradually warm to 24° C. and stirred for 1 h. Analysis indicated incomplete reaction. The reaction mixture was cooled again to 18° C. with an ice water bath and a fourth portion of H₂O₂ (30% wt in H₂O, 1.30 eq., 0.24 mL) was added over the period of 1 min at 18-20° C. The reaction mass was quenched with solid Sodium Metabisulfite (5.00 equiv., 8.00 mmol) at 24° C. under stirring. (solid sodium metabisulfite was added portionwise (0.2 eq. each), and after each addition of sodium metabisulfite, the suspension was stirred for 10 min). The presence of residual H₂O₂ by checked using a starch-iodine paper coloring test. c) Starch-iodine paper was made wet before addition of reaction mixture. Inorganic insoluble materials were removed by filtration on a sintered funnel. The collected solid was washed with CH₂Cl₂ (2×10 mL). The combined filtrate and washings were concentrated under reduced pressure to afford 1.453 g of solid crude material. The crude material was taken up in CH₂Cl₂ (25 mL) and stirred for 15 min at 24° C. The insoluble inorganic was again removed by filtration on sintered funnel and the filtrate was concentrated under reduced pressure till constant weight to afford of sticky yellow solid (0.25 g). This material was analyzed using quantitative 1HNMR in DMSO-d6 (using 1,3,5-trimethoxybenzene as an internal standard). The analysis indicated the title compound (7A, analytical data as reported above) had been formed in 29% Chemical yield.

1. A process for the preparation of a compound of formula (I) 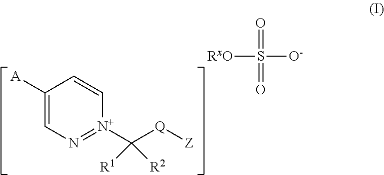
wherein
A is a 6-membered heteroaryl selected from the group consisting of formula A-I to A-VII below 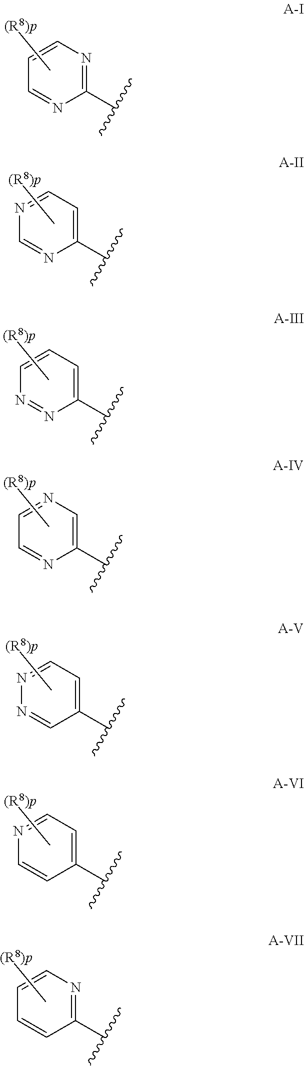
wherein the jagged line defines the point of attachment to the remaining part of a compound of formula (I), p is 0, 1 or 2; and
R^{x} is hydrogen or C₁-C₆alkyl;
R¹ is hydrogen or methyl;
R² is hydrogen or methyl;
Q is (CR^{1a}R^{2b})ₘ;
m is 0, 1 or 2;
each R^{1a} and R^{2b} are independently selected from the group consisting of hydrogen, methyl, —OH and —NH₂;
Z is selected from the group consisting of —CN, —C(S)OR¹⁰, —C(S)NR⁶R⁷, —C(S)SR¹⁰, —CH₂OR³, —CH(OR⁴)(OR^{4a}), —C(OR⁴)(OR^{4a})(OR^{4b}), —C(O)OR¹⁰, —C(O)NHCN, —C(O)NR⁶R⁷, —C(O)NHS(O)₂R¹² and —S(O)₂OR¹⁰; or
Z is selected from the group consisting of a group of formula Zₐ, Z_{b}, Z_{c}, Z_{d}, Zₑ and Z_{f} below 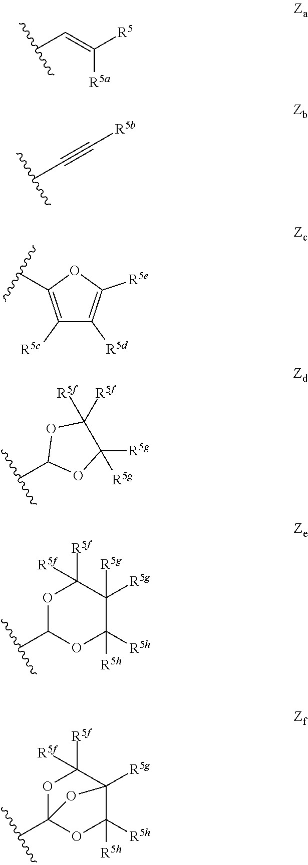
wherein the jagged line defines the point of attachment to the remaining part of a compound of formula (I); and
R³ is hydrogen or —C(O)OR^{10a};
each R⁴, R^{4a} and R^{4b} are independently selected from C₁-C₆alkyl;
each R⁵, R^{5a}, R^{5b}, R^{5c}, R^{5d}, R^{5e}, R^{5f}, R^{5g} and R^{5h} are independently selected from hydrogen and C₁-C₆alkyl;
each R⁶ and R⁷ are independently selected from hydrogen and C₁-C₆alkyl;
each R⁸ is independently selected from the group consisting of halo, —NH₂, methyl and methoxy;
R¹⁰ is selected from the group consisting of hydrogen, C₁-C₆alkyl, phenyl and benzyl;
R^{10a} is selected from the group consisting of hydrogen, C₁-C₆alkyl, phenyl and benzyl; and
R¹² is selected from the group consisting of methyl, —NH₂, —N(CH₃)₂ and —NHCH₃;
said process comprising:
reacting a compound of formula (II): 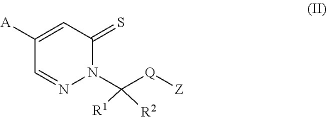
wherein A, R¹, R², Q and Z are as defined above, in a suitable reaction medium comprising a desulfurization agent, to give a compound of formula (I).

2. A process according to claim 1, wherein the compound of formula (I) is further subjected to a salt exchange to give a compound of formula (Id), 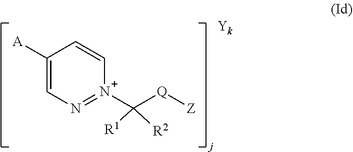
wherein Y represents an agronomically acceptable anion and j and k represent integers that may be selected from 1, 2 or 3, and A, R¹, R², Q and Z are as defined in claim 1.

3. A process according to claim 2, wherein the compound of formula (Id) is a compound of formula (Id-I), 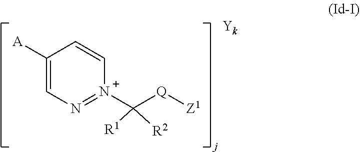
wherein A, R¹, R² and Q are as defined in claim 1, Z¹ is selected from the group consisting of —CN, —C(O)OR¹⁰, —C(O)NH₂ and —S(O)₂OR¹⁰, and R¹⁰ is selected from the group consisting of C₁-C₆alkyl, phenyl and benzyl;
and hydrolysing said compound of formula (Id-I) to a compound of formula (Ie), 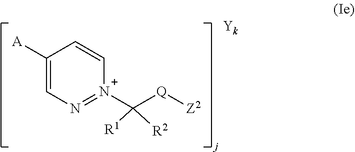
wherein A, R¹, R² and Q are as defined in claim 1 and Z² is —C(O)OH or —S(O)₂OH.

4. A process according to claim 1, wherein the compound of formula (I) is a compound of formula (Ib), 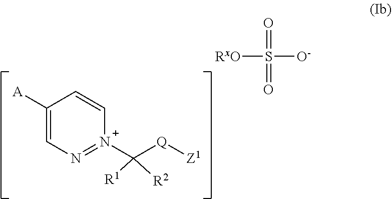
wherein A, R^{x}, R¹, R² and Q are as defined in claim 1, Z¹ is selected from the group consisting of —CN, —C(O)OR¹⁰, —C(O)NH₂ and —S(O)₂OR¹⁰, and R¹⁰ is selected from the group consisting of C₁-C₆alkyl, phenyl and benzyl;
and hydrolysing said compound of formula (Ib) to a compound of formula (Ic), 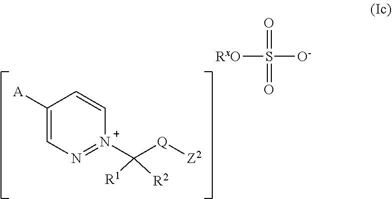
wherein A, R^{x}, R¹, R² and Q are as defined in claim 1 and Z² is —C(O)OH or —S(O)₂OH.

5. A process according to claim 4 wherein the compound of formula (Ic) is further subjected to a salt exchange to give a compound of formula (Ie), 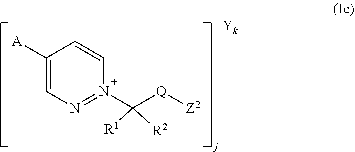
wherein Y represents an agronomically acceptable anion and j and k represent integers that may be selected from 1, 2 or 3, and A, R¹, R² and Q are as defined in claim 1 and Z² is —C(O)OH or —S(O)₂OH.

6. A process according to claim 1, wherein Y is chloride and j and k are 1.

7. A process according to claim 1, wherein R¹ and R² are hydrogen and R^{1a} and R^{2b} are hydrogen.

8. A process according to claim 1, wherein R^{x} is hydrogen.

9. A process according to claim 1, wherein m is 1.

10. A process according to claim 1, wherein p is 0.

11. A process according to claim 1, wherein A is selected from the group consisting of formula A-Ia to A-IIIa below, 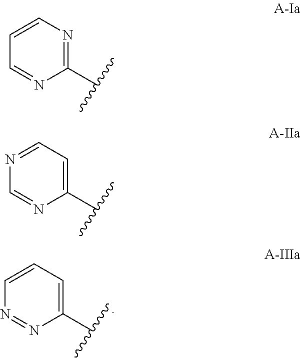 [SMILES: *c1ncccn1 ; *c1ccncn1 ; *c1cccnn1]
wherein the jagged line defines the point of attachment to the remaining part of a compound of formula (I).

12. A process according to claim 1, wherein Z is selected from the group consisting of —CN, —C(O)OR¹⁰, —C(O)NH₂ and —S(O)₂OR¹⁰.

13. A process according to claim 1, wherein the suitable reaction medium further comprises an acid.

14. A process according to claim 1, wherein the desulfurization agent is a peroxide.

15. A compound of formula (I) 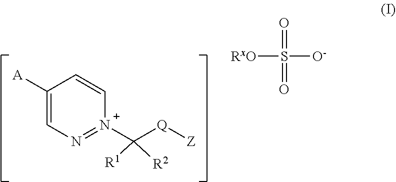
wherein A, R^{x}, R¹, R², Q and Z are as defined in claim 1.

16. A compound of formula (II) 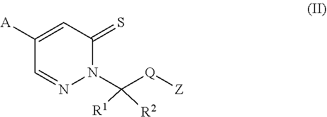
wherein A, R¹, R², Q and Z are as defined in claim 1.

17. A process according to claim 1 wherein the compound of formula (II) is produced by:
(i) reacting a compound of formula (III) 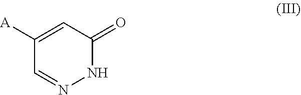
with a suitable alkylating agent to give a compound of formula (IV) 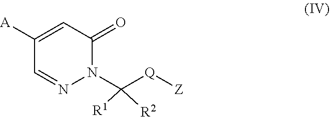
wherein A, R¹, R², Q and Z are as defined in claim 1, and
(ii) reacting the compound of formula (IV) with a sulfurizing agent to give a compound of formula (II)

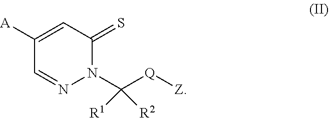

18. A compound of formula (IV) 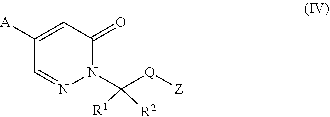
wherein A is a 6-membered heteroaryl selected from the group consisting of formula A-I to A-V and p, R¹, R², R⁸, Q and Z are as defined in claim 1.

19. Use of a compound of formula (III-I) for preparing a compound of formula (I) 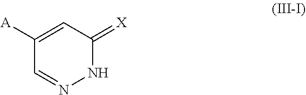
wherein X is S or O and A is as defined in claim 1.

20. A compound of formula (III-I)a 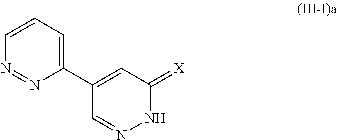
wherein X is S or O.
